(19) United States
(12) Patent Application Publication
(10) Pub. No.: US 2024/0006025 A1
(43) Pub. Date: Jan. 4, 2024

(54) METHODS AND SYSTEMS FOR GENERATING REGULATORY ELEMENTS

(71) Applicant: MONSANTO TECHNOLOGY LLC, Saint Louis, MO (US)

(72) Inventor: Jose Juan Tapia Valenzuela, Saint Louis, MO (US)

(21) Appl. No.: 18/215,789

(22) Filed: Jun. 28, 2023

Related U.S. Application Data
(60) Provisional application No. 63/358,085, filed on Jul. 1, 2022.

(51) Int. Cl. G16B 30/00; G16B 40/20

(52) U.S. Cl. CPC G16B 30/00; G16B 40/20

(57) ABSTRACT

Example systems and methods for use in generating synthetic regulatory elements are disclosed. One example computer-implemented method includes identifying, by a computing device, an input sequence associated with a regulatory element as a start sequence and calculating a score for the start sequence, based on a scoring function. The method also includes initializing N iterations, at desired parameters, and for each of the N iterations: (i) altering at least one nucleotide in an input sequence for the iteration; (ii) calculating a score for the altered sequence; (iii) advancing the altered sequence to a next iteration based on at least the calculated score for the altered sequence and a threshold; and (iv) identifying the altered sequence as an output sequence for the N iterations when the calculated score for the altered sequence indicates an enhancement over the input sequence and the iteration is equal to N.

### CROSS-REFERENCE TO RELATED APPLICATION

This application claims the benefit of, and priority to, U.S. Provisional Application No. 63/358,085, filed Jul. 1, 2022. The entire disclosure of the above application is incorporated herein by reference.

### FIELD

The present disclosure generally relates to methods and systems for use in plant biotechnology, and in particular to methods and systems for generating regulatory elements and, more particularly, synthetic regulatory elements, such as, for example, promotors, introns, and related polynucleotides, transgenic cells, and transgenic organisms.

### BACKGROUND

This section provides background information related to the present disclosure which is not necessarily prior art.

The production of transgenic cells and organisms through incorporation of heterologous gene(s) is routinely practiced by molecular biologists. Methods for incorporating an isolated nucleotide sequence into an expression cassette, producing transformation vectors, and transforming many types of cells and organisms are well known. However, the regulation or control of the gene's expression can be critical in the development of transgenic cells and organisms for commercial use. For example, in transgenic plants containing a heterologous gene conferring tolerance to a herbicide, it may be beneficial for the heterologous gene to be expressed in a temporal and spatial manner, for instance, corresponding to when the plant is exposed to the herbicide, and to what parts of the plant the herbicide normally exerts certain effects.

### SUMMARY

This section provides a general summary of the disclosure, and is not a comprehensive disclosure of its full scope or all of its features.

Example embodiments of the present disclosure generally relate to computer-implemented methods for use in generating regulatory elements (e.g., synthetic regulatory elements). In one example embodiment, such a method generally includes: identifying, by a computing device, an input sequence associated with a regulatory element as a start sequence; calculating, by the computing device, a score for the start sequence, based on a scoring function; initializing N iteration(s) at at least one parameter, where N is an integer; for each of the N iterations: (i) altering at least one nucleotide in an input sequence for the iteration; (ii) calculating a score for the altered sequence based on the scoring function; (iii) advancing the altered sequence to a next iteration based on the score(s), a probability function, and/or a predefined threshold (e.g., advancing the altered sequence to a next iteration in response to an acceptance probability function and/or the calculated score for the altered sequence satisfying a threshold and/or being greater than the calculated score for the start sequence and/or the iteration being less than N); and (iv) identifying the altered sequence as an output sequence for the N iterations when the calculated score indicates an enhancement over the input sequence and the iteration is equal to N; and after the N iterations, directing the output sequence to a validation phase, whereby the sequence defining the regulatory element is synthesized.

Example embodiments of the present disclosure also generally relate to systems for use in generating regulatory elements (e.g., synthetic regulatory elements). In one example embodiment, such a system generally includes a computing device configured to: identify an input sequence associated with a regulatory element as a start sequence; calculate a score for the start sequence, based on a scoring function; initialize N iteration(s) at at least one parameter, where N is an integer; for each of the N iterations: (i) alter at least one nucleotide in an input sequence for the iteration; (ii) calculate a score for the altered sequence based on the scoring function; (iii) advance the altered sequence to a next iteration based on the score(s), a probability function, and/or a predefined threshold (e.g., advance the altered sequence to a next iteration in response to an acceptance probability function and/or the calculated score for the altered sequence satisfying a threshold and/or being greater than the calculated score for the start sequence and/or the iteration being less than N); and (iv) identify the altered sequence as an output sequence for the N iterations when the calculated score indicates an enhancement over the input sequence and the iteration is equal to N; and after the N iterations, direct the output sequence to a validation phase, whereby the sequence defining the regulatory element is synthesized.

Example embodiments of the present disclosure also generally relate to non-transitory computer-readable storage media including computer-executable instructions that, when executed by at least one processor, cause the at least one processor to generate regulatory elements (e.g., synthetic regulatory elements). In one example embodiment, such a non-transitory computer-readable storage medium includes instructions that, when executed by at least one processor, cause the at least one processor to: identify an input sequence associated with a regulatory element as a start sequence; calculate a score for the start sequence, based on a scoring function; initialize N iteration(s) at at least one parameter, where N is an integer; for each of the N iterations: (i) alter at least one nucleotide in an input sequence for the iteration; (ii) calculate a score for the altered sequence based on the scoring function; (iii) advance the altered sequence to a next iteration based on the score(s), a probability function, and/or a predefined threshold (e.g., advance the altered sequence to a next iteration in response to an acceptance probability function and/or the calculated score for the altered sequence satisfying a threshold and/or being greater than the calculated score for the start sequence and/or the iteration being less than N); and (iv) identify the altered sequence as an output sequence for the N iterations when the calculated score indicates an enhancement over the input sequence and the iteration is equal to N; and after the N iterations, direct the output sequence to a validation phase, whereby the sequence defining the regulatory element is synthesized.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, are not all possible implementations, and are not intended to limit the scope of the present disclosure.

FIG. 1 illustrates an example system of the present disclosure suitable for use in generating synthetic regulatory elements, based on probability associated with gene expression;

FIG. 2 illustrates an example feature plot associated with feature extraction, which may be implemented in connection with the system of FIG. 1;

FIG. 3 is a block diagram of an example computing device that may be used in the system of FIG. 1; and

FIG. 4 illustrates an example method, which may be implemented in connection with the system of FIG. 1, for generating one or more synthetic regulatory elements.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Ability to control the expression of transgenes, by certain regulatory elements, is limited. The regulatory elements generally include those that are naturally occurring, and synthetic regulatory elements, which are made through complex biological experimentation, editing, and/or testing of the expressions, etc.

Uniquely, the systems and methods herein permit specific, novel or synthetic regulatory elements to be defined, and employed to enhance expression of traits in transgenic cells, organisms (including viruses and viral vectors), and polynucleotides, etc. The synthetic regulatory sequences are defined to satisfy gene expression objectives, including the ability to stack a plurality of heterologous genes (e.g., a “gene stack” or “stack”) for expression in a single cell, while avoiding gene silencing or reduced expression levels. In connection therewith, the systems and methods herein permit biological understanding of certain factors involved for a particular gene expression pattern, and rely on analysis of genomic and/or phenotypic data. In this manner, various advantages are provided including, for example, without limitation, (1) providing a source of unique synthetic regulatory elements; (2) providing expression patterns, regulation, and characteristics that are not available from naturally occurring regulatory elements; (3) limiting and/or alleviating gene silencing issues; and/or (4) providing additional compact synthetic regulatory sequences, etc.

The instant application includes subject matter that may be related to the subject matter included in Applicant's following patent applications: U.S. Provisional Patent Appl. No. 61/529,001, filed Aug. 30, 2011; U.S. Provisional Patent Appl. No. 61/535,109, filed Sep. 15, 2011; U.S. Provisional Patent Appl. No. 61/535,117, filed Sep. 15, 2011; U.S. patent application Ser. No. 13/599,254, filed Aug. 30, 2012; U.S. patent application Ser. No. 13/599,255, filed Aug. 30, 2012; U.S. patent application Ser. No. 15/408,402, filed Jan. 17, 2017; and U.S. patent application Ser. No. 17/165,734, filed Feb. 2, 2021. The entire disclosure of each of the above applications is incorporated herein by reference. In citing these applications, Applicant does not waive the confidentiality provisions of 35 U.S.C. § 122.

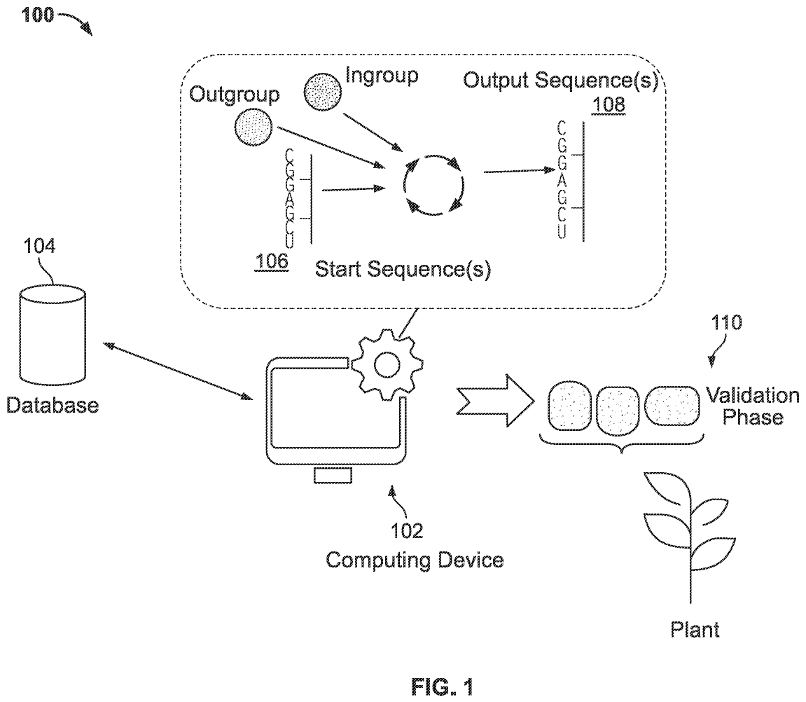

With that said, FIG. 1 illustrates an example system 100 for generating synthetic regulatory elements, in which one or more aspects of the present disclosure may be implemented. Although, in the described embodiment, parts of the system 100 are presented in one arrangement, other embodiments may include the same or different parts arranged otherwise depending, for example, on accessibility of the specific data, distribution of the operations associated with the disclosure, etc.

As shown in FIG. 1, the system 100 generally includes a computing device 102 and a database 104, where the database 104 is coupled in communication with the computing device 102. It should be appreciated that the database 104 may be separate (either physically or logically) from the computing device 102, as shown in FIG. 1, or alternatively, included, in whole or in part, in the computing device 102 in other embodiments.

The computing device 102 is configured to start from one or more start sequences 106, as is shown in FIG. 1. The computing device 102 is configured to evaluate the start sequence(s) 106, based on, for example, a scoring function, and then to alter the start sequence(s) 106, as described in more detail below, to generate one or more output sequences 108. The computing device 102 is configured to then evaluate the altered sequence(s) (or output sequence(s) 108), and to continue to alter and evaluate the sequence(s). In the end, the computing device 102 is configured to identify one or more output sequences 108, based on the relative evaluation of the start/altered sequence(s). The output sequence(s) 108 is representative of a synthetic regulatory element, which is applicable to, for example, plants, animals, algae, fungi, bacteria, or viruses, etc. Once the output sequence(s) 108 is identified, the sequence(s) 108 of the synthetic regulatory element is exposed to a validation phase 110 of the system 100. In this embodiment, the validation phase 110 includes the transformation of the sequence(s) 108 representing the synthetic regulatory element into a plasmid, for example, and the transformation of a gene of interest within the plasmid, to take advantage of the expression regulation by the generated synthetic regulatory element, etc.

As used herein, the term “regulatory element” refers to a nucleotide sequence that is involved in controlling gene expression in an organism of interest. Genetic regulatory elements include, for example, promoters, leaders (also known as 5′UTRs), enhancers, introns, transcription termination regions (or 3′UTRs), polyadenylation signals, and chromatin control elements, or other sequences affecting RNA transcription, mRNA processing, RNA turnover or abundance, or translation of RNA, etc. The regulatory elements may include 5′-untranslated regions (5′UTRs) or parts thereof, 3′-untranslated regions (3′UTRs) or part thereof, or intronic sequences, etc. It is recognized that a regulatory element may include one or more additional regulatory elements such as, for example, an enhancer, etc. It is further recognized that regulatory elements may perform in concert with other regulatory elements to control the regulation of an operably linked gene of interest. Moreover, it is recognized that an enhancer can, at times, be separated from the transcribed region of a gene of interest by 1, 2, 3, or more kilobases of DNA.

In this example embodiment, the database 104 includes hundreds, thousands, tens of thousands, or hundreds of thousands, or more or less, genes for a variety of different organisms. The database 104 includes the genetic sequences of the genes. Additionally, the database 104 identifies the specific regulatory elements included in the genes, which control the expression of the genes. For each gene in the database 104, the database may include expression data, which indicates, for example, that the regulatory elements included in the gene are effective in expressing the gene in the organism. Consequently, then, the database 104 may separate the genes and/or regulatory elements based on the expression thereof.

In this manner, for a specific organism and a specific gene to be expressed, the database 104 may include a set of known regulatory elements having one or more selected gene expression properties, and a set of known regulatory elements that do not have the one or more selected gene expression properties. These sets of known regulatory elements may be understood to be training sets for the use(s) as described herein, etc. The training set(s) of regulatory elements may further include one species or multiple species or genus (or virus family).

It should be understood that a set of regulatory elements having the one or more selected gene expression properties may include all known sequences (as described above) from one or more selected species or genera (or virus families), and which are known to exhibit the one or more selected properties (or include the feature(s) associated with the one or more gene expression properties). The computing device 102 herein may be configured to proceed based on the set of regulatory elements or a subset of these sequences. The set of regulatory elements may include at least about 10 regulatory elements up to about 10,000 or more (without limitation). Preferably, in one or more example embodiments, the set of regulatory elements includes from about 25 to about 300 elements. In certain embodiments, the set of regulatory elements having the one or more selected gene expression properties may include at least about 25 elements, at least about 30 elements, at least about 35 elements, or at least about 40 elements, or at least about 100 elements. In other embodiments, the computing device 102 may be configured to operate on at least about 300, at least about 350, or at least about 400 of such regulatory elements. These sets of existing sequences can be obtained from the various publicly available genomes.

It is further recognized that the number of genes will vary depending on a number of factors including, for example, the choice of a target organism, the genetic regulatory element, and the word or oligomer window length. Generally, a sufficient number of sequences should be used to provide enough statistical power.

In addition to the genes and/or regulatory elements, the database 104 may include other data related to the genes and/or regulatory elements, such as, for example, features of the genes and/or regulatory elements (e.g., GC content, deleterious motifs, etc.), etc., based on prior knowledge of the genes and/or regulatory elements, etc. The other data is provided in more detail in the description of the computing device 102, below.

In this example embodiment, the system 100 employs one or more feature extraction techniques, whereby certain features of sequences associated with selected or desired gene expression properties may be identified (e.g., a list of motifs and sequence features to template in (or out) of target elements, sequence features that correlate with a target of interest (or target element if interest), etc.).

In one example feature extraction technique, the computing device 102 may be configured to determine, for a set of regulatory elements, values for a listing of features of the regulatory elements from the database 104. The features may include, for example, different patterns of base pairs (e.g., GC content, etc.), which may be position dependent or position independent; k-mer frequency (e.g., the frequency at which a given oligomer window (e.g. word) appears in a nucleotide or protein sequence, etc.); RNA secondary structure; codon frequency; other specialized features (e.g., codon stability coefficients, codon adaptation indices, presence of intronic motifs, etc.) etc.

Specifically, in this example, a user (e.g., a scientist, breeder, other user, etc.) associated with the system 100 selects at least one organism (e.g., a target plant, etc.), and one or more specific properties of gene expression (e.g., one or more selected gene expression properties, etc.). Examples of gene expression properties include, but are not limited to, expression level (e.g., strong expression, weak expression, etc.), inducible expression (e.g., stress inducible expression, hormone or chemical inducible expression, etc.), temporal control of gene expression and spatial control of gene expression in the selected organism. In some embodiments, the selected gene expression property(ies) may include constitutive expression (e.g., high or low constitutive expression, etc.), cell specific expression, tissue specific expression, or organ specific expression. The selected gene expression property(ies) in some embodiments may be expressed in response to biotic stress (e.g., fungal, bacterial and viral pathogens, insects, herbivores and the like, etc.) and/or abiotic stress (e.g., wounding, drought, cold, heat, high nutrient levels, low nutrient levels, metals, light, herbicides, pesticides, other synthetic chemicals, and the like, etc.). In further embodiments, the selected property(ies) of gene expression may be developmentally controlled in one or more of plant stems, leaves, roots, and seeds. In one embodiment, the selected pattern of expression may be a constitutive expression, such as a constitutive expression in plant roots, a constitutive expression in all the tissues of the roots, a constitutive expression in the meristem, etc.

In connection therewith, the computing device 102 is configured to access the database 104, and specifically, a first set of regulatory elements (e.g., nucleotides or amino acids, etc.) for the selected organism, which includes the selected gene expression property(ies), and also a second set of regulatory elements for the selected organism, which does not include the selected gene expression property(ies). The computing device 102 is configured to access a listing of features of interest (e.g., defined by the user, etc.) in the database 104 and to assess the specific features of interest for the first and second sets of regulatory elements.

For instance, certain features may include a frequency of a specific motif of length k. As such, the computing device 102 is configured to extract the frequency at which all motifs of a length k, or k-mers, are included in the regulatory elements. In general, depending on the number of motifs searched within the elements, the features may include, for example, 4^{k} different features. As apparent, the selection of the value k may be related to the performance capabilities of the computing device 102, or not. As other potential feature(s), the computing device 102 may be configured to determine the occurrence of specific motifs in the regulatory elements. For example, the computing device 102 may be configured to access a set of lists of deleterious motifs (e.g., as defined by the user, the database 104, etc.), and determine the occurrence of the deleterious motifs in each of the regulatory elements, in one or more scenarios (like a list of μRNA targeting sites), etc. The specific motifs may be of varying lengths, or arbitrary lengths, as suitable to a specific implementation and/or embodiment, etc.

Further features may include one or more sequence metrics, structural metrics and/or gene specific metrics. For instance, the computing device 102 may be configured to determine the GC content of each regulatory element. Other sequence metrics may include, for example, codon stability coefficients, codon adaptation indices, codon usage indices, GC3 content (e.g., GC content of every third nucleotide, etc.), free energy of the predicted RNA secondary structure, number of unpaired bases in the predicted RNA secondary structure, etc. It should be appreciated that other sequence metrics may define features in other system embodiments, etc. In another example, the computing device 102 may be configured to determine structural metrics, which may include metrics associated with features that tend to model or approximate secondary structure of the RNA transcript resulting from the sequence. And, also, in various examples, the computing device 102 may be configured to determine gene specific metrics, which may include frequencies of different codons, a codon adaptation index (e.g., a correlation metric between codon in the input sequence vs its usage in a reference set of high expressing genes, etc.), a codon stability coefficient, etc.

It should be appreciated that each of the above features may be targeted to the specific regions in the gene and/or regulatory element (e.g., close to 5′ or 3′ ends, proximity to a transcription start site, etc.).

Based on the above, the computing device 102 may further be configured to generate a feature matrix, which is generally defined as a n×m matrix, where n is the number of regulatory elements, and m is the number of features included in the listing accessed in the database 104. As such, the feature matrix may comprise one or multiple rows that include a listing of features such as sequence GC content, sequence predicted secondary structure based energy, frequency of a given k-mer, etc. for a given regulatory element. That said, in some implementations of the present disclosure, the feature matrix may not be required or generated.

Consequently, the feature matrix (when generated) provides certain dimensionality, where m>>n, such that feature reduction may be desirable in various example embodiments (although such feature reduction is not required in all example embodiments of the present disclosure). In such embodiments where feature reduction is desirable, the computing device 102 may be configured, optionally, to provide for feature reduction through various techniques.

In one feature reduction example, the computing device 102 is configured to rely on information content-based reduction. In connection therewith, the computing device 102 is configured to measure variance of each individual feature when taken into isolation. For example, a feature for which there are only 0's in the matrix would be considered to be 0 variance, whereas a column with many different values would be considered high-variance across the regulatory elements included in the matrix. As such, the computing device 102 is configured, generally, to identify the high-variance columns as more likely to contain useful information for a machine-learning process. Consequently, in this example, the computing device 102 may be configured to select the top-k columns (features) with the most variance.

Additionally, or alternatively, in another feature reduction example, the computing device 102 is configured to rely on model-based reduction. In connection therewith, the computing device 102 is configured to rely on ranking of features, through certain learning algorithms, whereby weights are placed on certain features when defining an internal model. The computing device 102 is configured, then, to leverage the ranking by either selecting a single model's features (e.g., random forest, lasso-based selection, etc.), or polling a number of simple models and selecting for a consensus set of features.

Regardless of the feature reduction technique employed, if any, the computing device 102 is configured to then perform feature analysis in connection with the features, for example, included in the feature matrix, etc. In particular, in this example, the computing device 102 is configured to select for the strongest features that are either correlated or anti-correlated gene expressions of the organism. In particular, each of the features are subject to an analysis, whereby feature contribution may be interpreted.

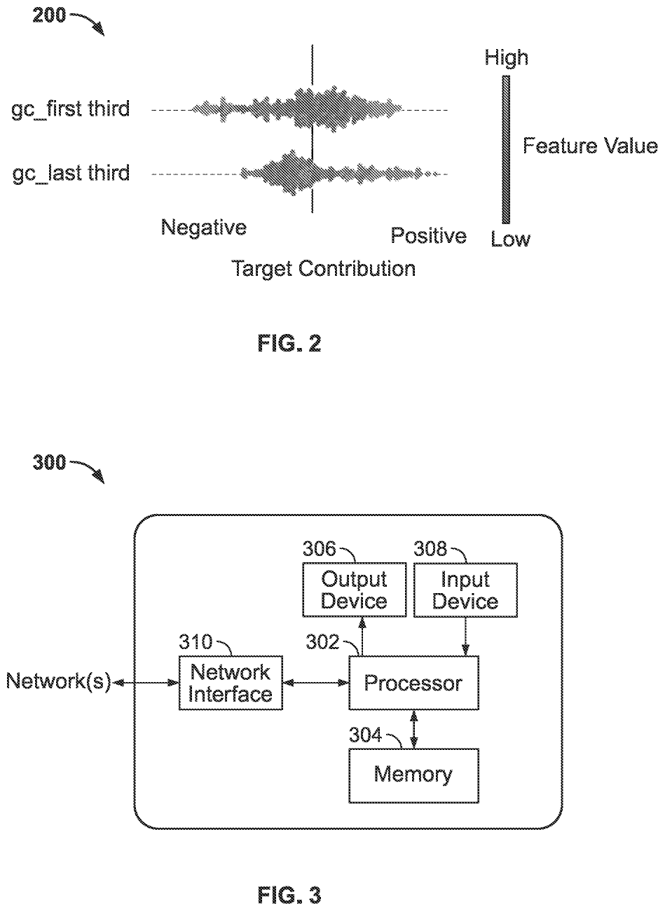

One example technique employed by the computing device 102 may include application of Shapley Additive Explanations, or SHAP, plots, as illustrated, for example, at 200 in FIG. 2. Consistent with SHAP, for example, an explanation model is generated whereby feature contributions are provided visually. As such, SHAP plots provide indicators, where each feature is assigned a SHAP value, which represents a change in the expected prediction in a prediction model when conditioning on that feature. With reference to FIG. 2, the shading is indicative of whether a given feature is high or low, and the position of the shaded indicator towards the left or right of the vertical axis 202 (e.g., target value, etc.) indicates whether the correlation of a feature value towards the target value is positive or negative. In this example, based on visual analysis of the SHAP plot in FIG. 2, high GC content, for instance, in the first third of a sequence is negatively correlated to the target value, and high GC content in the last third is positively correlated. In some examples, in addition to (or in place of) the visual analysis, a ranking system may be employed to evaluate the prevalence of different features using non-linear correlations (e.g., similar to those above, etc.) of the feature with the target class in order to discard features that are not significant (e.g., were the determined correlations are ordered, ranked, etc.).

In view of the above, the computing device 102 is configured to identify a set of motifs and/or metrics, and associated features, to be included in the scoring of potential regulatory elements (as described more below). As such, the development of an algorithm, by the computing device 102, may efficiently and accurately define the specific features from existing expression data as being of particular contribution to the expression of the gene in the organism. In doing so, the various features identified herein may then be accounted for in the given algorithm and thus directly accounted for in the related scoring, as desired (e.g., depending on the design of the given algorithm, etc.).

It should be appreciated that the identified set of motifs and/or features described above is/are stored in the database 104 for use in the operations described below. Consequently, the computing device 102 may be configured to select ingroup sets of motifs (as shown in FIG. 1) and outgroup sets of motifs (as shown in FIG. 1) based on correlation to gene expression. The computing device 102 is configured to then define ingroups and outgroups, where the ingroup motifs may include a set of motifs that correlate to expression of the selected gene in certain organisms, and the outgroup motifs may include all other motifs, or motifs specially associated with different gene expressions (e.g., a lack of gene expression, low gene expression, high gene expression, inducible gene expression, constitutive gene expression, tissue specific gene expression, etc.), or a positive effect on the sequence, or negative effect on the sequence (e.g., deleterious motifs, etc.), etc.

In another example, the computing device 102 may employ position-sensitive word set (POWRS) algorithm in connection with feature analysis/extraction. In particular, POWRS may be used for identifying features of sequences associated with selected or desired gene expression properties. For instance, POWRS uses position-specific enrichment of regulatory elements near transcription start sites to improve sensitivity, while also providing information about a preferred localization of those elements.

In still another example, the computing device 102 may employ one or more additional steps or processes in connection with feature analysis/extraction. For example, the computing device 102 may be configured to determine a frequency of short oligomer windows of predetermined length(s) in known sequences. As used herein, “oligomer window” refers to a short nucleotide sequence. Furthermore, “frequency” may refer to a count of the number of occurrences of each such oligomer window; or to the fraction or percentage of all oligomer windows which such count comprises; or to a ratio of such fractions between two sets of known sequences, and thus, reflecting the frequency “enrichment” of an oligomer window in one set relative to the other.

In certain embodiments, when determining position-dependent or position-independent enrichment of oligomer windows, the computing device 102 may be configured to determine enrichment with respect to a set of background elements (or a “second set”) that do not have (or are not predicted to have) the selected property. Generally, the second set of regulatory elements includes all or a portion of the class of regulatory elements in an organism. In some embodiments, the second set may include from about 20,000 to about 60,000 regulatory elements but in other embodiments the second set may include only a subset of these regulatory elements from the target organism. Typically, the second set includes at least about 100 regulatory elements. That said, it should be appreciated that, in certain other embodiments, a “simulated background” process may be used, whereby the second set of elements may be omitted. The simulated background approach can be used, for example, in the design of virus promoters. Briefly, the simulated background scheme involves determining the position-dependent enrichment of the oligomer windows in the first set of regulatory elements having the gene expression property (or properties), with respect to the total occurrence of the oligomer window in the set of regulatory elements.

It should be appreciated that various other techniques may be employed to correlate specific metrics to feature expressions in the set of elements.

That said, as disclosed in detail hereinafter, a scoring function may be implemented by the computing device 102 to calculate, for each oligomer window (or “word”) of a selected size in the sequence, a position-dependent or position-independent enrichment in the set of regulatory elements having the selected gene expression property (or properties). That is, an oligomer window size is selected (such as a 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, etc.), and each oligomer window in the sequence (or in a portion of the sequence) is analyzed for a position-dependent and/or position-independent enrichment in the set of regulatory elements with the selected property. An aggregate score may then be determined, which represents a probability that the sequence has the selected gene expression property (or properties) in a species of interest. Known algorithms may be employed to predict the likelihood that the nucleotide sequence has the selected property, such as Bayes' rule in some embodiments.

In certain embodiments, the computing device 102 is configured to construct a genetic regulatory element that can appear more than once in a gene of interest such as, for example, an intron. In such embodiments, the first set of genetic regulatory elements may include all introns that occur in a specified position (e.g., the first or last intron in a gene, etc.) and the second set of genetic regulatory elements may include all introns in the genome of the organism that fall outside of the specified position. In one embodiment, the first set of genetic regulatory elements includes first introns from highly expressed constitutive genes that occur in either the 5′UTR or the coding region and within 500 base pairs (bp) of a transcription start site (TSS), and the second set of nucleotide sequences then includes all non-first introns of all genes in the target organism.

The computing device 102 is configured to align the set of regulatory elements around one or more conserved sequence or “landmark” sequence for position-dependent analysis of enriched sequences. The conserved sequence(s) or landmark(s) may be, for example, a transcription start site (TSS), a TATA box, a transcription termination signal, a polyadenylation signal, a splice acceptor site, a splice donor site, or a branch site. In certain embodiments, the conserved sequence is a TSS or TATA box. In some embodiments, the landmark sequence includes the 5′ and/or 3′ end of the element, or other conserved motif(s) or sub-element(s) within the genetic element. However, any method of aligning the sequences known in the art can be used. For example, when the genetic regulatory element is an intron, the computing device 102 may be configured to align intron sequences on both 5′ and 3′ splice sites, and duplicate or truncate the middle sequence as needed to provide for appropriate length. In addition, negative motifs (e.g., motifs to exclude from the final sequence, etc.) may also be identified.

It should be appreciated that the computing device 102 may be configured to select an oligomer window or word length to use in comparing the sequences, where the oligomer window or word length includes the number of contiguous nucleotides in the oligomer window. For a given implementation, the word length may be fixed. The word length may be about 4, 5, 6, 7, 8, 9, 10, 11, 12, 13, 14, 15, 16, 17, 18, 19, 20, etc. For each word length x, there are 4× possible words, due to the possibility of an A, G, C, or T at each nucleotide position, although all words might not be represented in the nucleotide sequences of a set of genetic regulatory elements.

In general, as described (e.g., as part of feature extraction, etc.), different elements in the ingroup and outgroup may be aligned around a given landmark (since they potentially have differences in length and therefore need to be centered around something). Then, different properties may be extracted, as described.

As disclosed in various embodiments herein, the computing device 102 is configured to calculate, based on the scoring function, a position dependent and/or position independent score for a plurality of oligomer windows, and to determine a probability that the start or altered sequence will have the selected property based on an aggregate or factor of said position-dependent and/or position independent scores. The position-dependent enrichment of an oligomer window in the set of regulatory sequences with the selected property means that the oligomer sequence is enriched at the same position or a position defined as within ±200, or in some embodiments within ±100, or in some embodiments within ±30 nucleotides. In some embodiments, position-dependent enrichment is constrained to within ±20 nucleotides or within ±10 nucleotides.

In various embodiments, only part of the nucleotide sequence is analyzed for position-dependent enrichment of the oligomer window, since the predicted importance of the positioning may depend on the type of element or vary within an element. In other words, different parts of the training set herein may be treated differently depending on their position relative to various landmarks. For example, where the synthetic regulatory element is a promoter, the position-dependent enrichment of the oligomer windows may be less important at regions distant from the TSS or TATA box. Therefore, in some embodiments, the position-dependent enrichment of the oligomer windows may be determined in the set of regulatory elements with the selected property within at least the 20 bp region upstream and/or downstream from the TSS or TATA box. For example, relative to the TSS, a region comprising −50 to +20, or −100 to +20, or −200 to +20, or −50 to +50, or −100 to +50, or −200 to +50 may be analyzed for position-dependent enrichment of oligomer windows. In other embodiments, position-dependent enrichment is determined for at least about 50 bases, or at least about 100 bases upstream of the TSS or TATA Box. Other oligomer windows outside of these regions may be analyzed in a position-dependent or position-independent manner.

In some embodiments, the process maintains a level of sequence complexity or weights local sequence complexity, by inserting repeated base pairs, for example, such that the synthetic regulatory element approximates the sequence complexity (including locally in some embodiments) of the set of regulatory elements with the desired gene expression property (or properties). Sequence complexity can be defined by the GC or AT content, or defined by dinucleotide content (e.g., AA, AT, AC, AG, TT, TA, TC, TG, CC, CG, CT, CA, GG, GC, GA, and GT, etc.), or defined by the A, T, G, and/or C fractions. A separate score for local sequence complexity may be determined for various segments of the polynucleotide. Such segments may be at least 30 bp, and in some embodiments are at least 50 bp, or at least 100 bp, or at least 125 bp in length. In such embodiments, the computing device 102 may employ an algorithm (e.g., as part of an entropy element in scoring herein (e.g., Z₄(S) herein, etc.), etc.) to calculate local sequence complexities, and thereby constrain local sequence complexity to approximate the local sequence complexity of the elements having the selected property.

In iteratively or non-iteratively modifying the sequence, the computing device 102 may be configured to employ any suitable technique to modify the sequence. In some embodiments, for example, the computing device 102 may be configured to employ simulated annealing, while in other embodiment, the computing device 102 may be configured to employ other types of algorithms, including, without limitations, genetic algorithms, tabu search, simplex algorithm, steepest descent, conjugate gradients, and dynamic programming.

After the feature expression and recognition of the features and/or motifs, the computing device 102 is configured to rely on the same to generate a synthetic regulatory element. At the outset, the computing device 102 is configured to select the input sequence 106, from which the synthetic regulatory element is to be generated. Various different techniques may be employed to select the input sequence.

In various embodiments, synthetic regulatory elements are generated based on iterative modification of the sequence, and then scored through one or more scoring functions rather than by combining sequences from a defined group of sub-sequences. The scoring function(s), as described herein, may be probabilistic in nature and may be used to design sequences to be similar to members of a set of naturally occurring regulatory elements included in the first set of regulatory elements (e.g., having the desired one or more gene expression properties, etc.), however, the designed sequences have limited extended sequence homology with the naturally occurring sequences. In this manner, the scoring functions do not require predetermined knowledge of functional motifs, cis-elements, transcription factor binding sites, etc. Because of these characteristics, the configurations and processes described herein are widely applicable to both promoter and non-promoter regulatory elements, including, for example, introns and untranslated regions (UTRs), for which little or no functional motif information is available.

In connection therewith, as indicated above, the computing device 102 may be configured to obtain at least a first set of sequences of a genetic regulatory element or part thereof, wherein the first set of sequences is from a selected organism (e.g., a target organism), and each of the genes in the first set of genes is known or expected to be expressed in a desired manner in the target organism. The computing device 102 may further be configured to determine for the first set of sequences the frequency of each word of a pre-determined word length. Each word's position-dependent or position-independent enrichment may be determined as described herein. The computing device 102 may be further configured to define the output sequence 108 for the synthetic genetic regulatory element or part thereof by starting from the input sequence 106 and generating at least one modified sequence and then scoring the at least one modified sequence with a scoring function, in pursuit of an improved score.

In this example embodiment, in view of the above, the input sequence 106, may, for example, be a sequence from the first set of nucleotide sequences described above, a known sequence associated with the property, or a sequence that is generated using a scoring function described below.

The score of a sequence is derived from such a scoring function, as described herein, which indicates, for example, at least in part a similarity of the sequence to the first set of regulatory elements. The score is derived from the frequencies of the words in the first set of regulatory elements. Typically, the desired score is a score that is higher than the scores of about 1%, 5%, or 10% of the nucleotide sequences in the first set of regulatory elements. In some embodiments, the desired score is a score that is higher than the scores of about 20%, 25%, or 30% of the gene expression elements in the first set. In other embodiments, the desired score is a score that is higher than the scores of about 40%, 50%, 60% or more of the nucleotide sequences in the first set of nucleotide sequences. It should be appreciated that other score thresholds may be employed in other embodiments. It should further be appreciated that the computing device 102 may be configured to continue to generate and score additional related sequence(s) until a related sequence comprising a desired score (e.g., relative to a score threshold, etc.) is generated.

Thus, as is described in further detail below, the computing device 102 may be configured to determine: (i) the frequency of each word in a first set of genetic regulatory elements; (ii) the enrichment of each word in said genetic regulatory elements relative either to the occurrence of each word in a second set of genetic regulatory elements or to the frequency of the word over all positions in the first set of genetic regulatory elements (e.g., a second set of genetic regulatory elements is not used, etc.); (iii) the sequence entropy of the genetic regulatory element, in connection with the scoring function herein; and/or (iv) the local and/or global similarity of the sequence to the regulatory elements included in the set of regulatory elements, etc.

In particular, the computing device 102 may be configured to compare the nucleotide sequences from a first set (A) to the nucleotide sequences from a second background set (B) and to determine what features of the genetic regulatory elements of A are likely to contribute to the distinctive expression pattern of those genes or elements. For example, the genetic regulatory element of interest may be a promoter. Promoters from A and B are aligned, for example, relative to their TSSs, and the comparison may be performed in a position-specific manner, for example, as a function of the distance from the TSS. As a variation, the sequences may be aligned around a conserved element near the TSS, such as, for example, the TATA box. Specifically, at each position, it is determined if the word or oligomer window sequences (also referred to herein as “k-mers”, for example, 4 to 10 consecutive bases) are overrepresented in the genes of interest.

In this manner, the computing device 102 is configured to rely on features, which exist in the first set, but not the second set as an indicator of expression.

In this example embodiment, the computing device 102 is configured to select the input sequence 106, or input regulatory element, from a set of regulatory elements known to exhibit desired expression properties of the selected gene for the selected organism. For example, it should be appreciated that certain existing, naturally occurring regulatory elements from a source species or organism with the selected gene expression property (or properties) are generally understood from genomic data. For example, microarray or RNA-sequence analysis may be employed to quantify transcripts in cells and tissues of interest, with correlation of expression patterns to the cognate genetic regulatory elements. And, the target species may be one or more plant, and various types and species of target plants are described elsewhere herein. Genetic data from these target species may be used for preparing synthetic regulatory elements.

In other embodiments, though (as generally indicated above), the computing device 102 may be configured to generate the input sequence, or input regulatory element (e.g., synthetic regulator element), based on a set of regulatory elements having a selected property of gene expression.

Regardless of the manner in which the input sequence 106 is identified, selected or compiled, the computing device 102 is configured to then score the input sequence 106 based on the scoring function, as described more below, and alter or modify the sequence (e.g., provide instructions for doing so, etc.). The computing device 102 is configured to then score the modified sequence, and to repeat, as needed in an iterative or non-iterative manner. In this manner, the computing device 102 is configured to define a sequence characterized in a suitable score, or a statistically significant score, whereby the sequence is likely to have the selected one or more gene expression properties. In this context, the term “statistically significant” means that the sequence contains a position-dependent or position-independent enrichment of oligomer window sequences found in the set of regulatory sequences having the selected one or more gene expression properties, and that the level of enrichment is unlikely to occur by chance. For example, a statistically significant score may have a p-value of about 0.05 or less, or a p-value of about 0.005 or less, etc.

In connection with the above, in one or more example embodiments, the computing device 102 is configured to produce a nucleotide sequence S that approximately maximizes the probability of expression pattern E in Equations (1)-(4) below, for example, to (approximately) maximize P(E|S). For convenience, k is used to denote both the length of the short sequences (typically 4-10 bp) and the sequences themselves (e.g., GCCCA, etc.). And, G represents the union of sequence sets A and B. For each position i relative to the TSS, and each k-mer k, G_{k,i} includes those sequences in G that contain k at position i. The k-mer at i and the k-mer at i+1 overlap each other by k−1 bases. Also, Gᵢ includes the sequences in G that contain position i (as regulatory elements differ in length, certain G may not include position i). Given the above, then, the scoring function, in this example, includes P(E|k,i) as the probability that a sequence having k at position i will display expression pattern E:

$$P ( E \vert i ) = P ( E ) = \frac{‖ A ‖}{‖ G ‖} \qquad ( 1 )$$

$$P ( k \vert i ) = \frac{‖ {G}_{k , i} ‖}{‖ {G}_{i} ‖} \qquad ( 2 )$$

$$P ( k \vert E , i ) = \frac{‖ {A}_{k , i} ‖}{‖ {A}_{i} ‖} \qquad ( 3 )$$

$$( 4 )$$

$$P ( E \vert k , i ) = \frac{P ( k \vert E , i ) P ( E \vert i )}{P ( k \vert i )} = \frac{\frac{‖ {A}_{k , i} ‖}{‖ {A}_{i} ‖} \cdot \frac{‖ A ‖}{‖ G ‖}}{\frac{‖ {G}_{k , i} ‖}{‖ {G}_{i} ‖}} = \frac{‖ {A}_{k , i} ‖}{‖ {A}_{i} ‖} \cdot \frac{‖ A ‖}{‖ G ‖} \cdot \frac{‖ {G}_{i} ‖}{‖ {G}_{k , i} ‖}$$

The probability P(E|S) of sequence S giving expression pattern E can be estimated by assuming the position-wise probabilities are independent and multiplying them together. This procedure is similar to a naive Bayes classifier. These probabilities can be normalized by the base probability of expression pattern E and log-transform them, yielding a score Z₁(S) that is greater than zero if sequence S is more likely than average to display pattern E, and less than zero if S is less likely than average to display pattern E, as expressed in example Equation (5).

$${Z}_{1} ( S ) = {\sum }_{i ∈ S} log \frac{P ( E \vert k , i )}{P ( E )} = {\sum }_{i ∈ S} log ( \frac{‖ {A}_{k , i} ‖}{‖ {A}_{i} ‖} \cdot \frac{‖ {G}_{i} ‖}{‖ {G}_{k , i} ‖} ) \qquad ( 5 )$$

where k is understood to be k_{S,i}, the k-mer at position i of sequence S. Thus, the term inside the logarithm is merely the fold enrichment of k in the genes of interest compared to the genome as a whole.

In various embodiments, additional terms may be desired. First, it should be appreciated that the oligomer window length of the k-mers may be indicative of how informative the k-mer is, where, for example, the longer k-mers are generally more informative, but it may be appreciated that for certain transcription factors, for example, that length of a given nucleotide signature may desirably be consistent with a window length of the k-mers to promote sufficient information without additional spurious information. That said, there are typically substantially more possible k-mers than genes of interest, meaning ∥A_{k,i}∥ is rarely greater than 1, and is often zero. For instance, there are 4096 possible 6-mers, and 65,536 possible 8-mers. Second, some k-mers are inherently uncommon in the genome, such that a limited number of occurrences in A leads to a high apparent enrichment.

In connection with the above, the scoring function may be modified by counting occurrences of k over a local oligomer window, instead of just at position i. The count is done as a kernel density estimate with a cosine kernel, with half-width at half-height of w (w=10 bps, or 5 bps, or 15 bps or otherwise, etc.), as shown in example Equation (6).

$$〈 {A}_{k , i} 〉 = \frac{1}{2 w} {\sum }_{j = i - 2 w}^{i + 2 w} \frac{cos ( \frac{π ( i - j )}{2 w} ) + 1}{2} ‖ {A}_{k , j} ‖ \qquad ( 6 )$$

One skilled in the art will recognize that other kernels (e.g., Gaussian, triangular, square, etc.) or methods (e.g., standard, smoothed, or averaged-shifted histograms, etc.) may be used in other embodiments.

What's more, the scoring function may be modified by adding pseudo-counts p to the actual observations; this corresponds to presuming a uniform distribution as the Bayesian prior. For most of the embodiments disclosed herein, p is 20, or alternatively, may include values from 10 to 50. Each of these modifications are provided in example Equation (7) directly below for Z₂ (S).

$${Z}_{2} ( S ) = {\sum }_{i ∈ S} log \frac{P ( E \vert k , i )}{P ( E )} = {\sum }_{i ∈ S} log ( \frac{‖ {G}_{i} ‖}{‖ {A}_{i} ‖} \cdot \frac{〈 {A}_{k , i} 〉 + \frac{‖ {A}_{i} ‖}{‖ {G}_{i} ‖} ρ}{〈 {G}_{k , i} 〉 + ρ} ) \qquad ( 7 )$$

Notwithstanding the above, optionally, the scoring function may be modified to limit contribution to the k-mer count from individual genes, while still smoothing counts over a local oligomer window. Such a modification may be used to account for genes containing the same k-mer many times in a small region, which may occur in situations in which a k-mer overlaps the preceding one by (k−1) out of k bases. In these cases, as little as one gene with a long repeat may cause an apparent enrichment of a k-mer like “GGGGGG”. The modification includes example Equation (8) below.

$$= \frac{1}{2 w} {\sum }_{a ∈ A} min ( 1 , {\sum }_{j = i - 2 w}^{i + 2 w} \frac{cos ( \frac{π ( i - j )}{2 w} ) + 1}{2} ‖ {a}_{k , j} ‖ ) \vert \qquad ( 8 )$$

In Equation (8), ∥a_{k,j}∥=1 if gene “a” contains k-mer k at position j, and 0 otherwise. By this above, the scoring function is modified to be the scoring function, Z₃(S), as shown in example Equation (9).

$${Z}_{3} ( S ) = {\sum }_{i ∈ S} log \frac{P ( E \vert k , i )}{P ( E )} = {\sum }_{i ∈ S} log ( \frac{‖ {G}_{i} ‖}{‖ {A}_{i} ‖} \cdot \frac{+ \frac{‖ {A}_{i} ‖}{‖ {G}_{i} ‖} ρ}{+ ρ} ) \qquad ( 9 )$$

It should be appreciated that, from the above, altered sequences that provide improved scores through Z₃(S) should be likely to drive gene expression following pattern E.

However, simply improving scores for Z₃(S) does not assure that a sequence will be promoter-like: there may be certain features or properties that are common to all promoters, and Z₃(S) does not detect such features. In practice, a sequence that scores higher for Z₃(S) will consist almost exclusively of k-mers that are actually observed with significant frequency in natural promoters. However, it was observed that for some species (e.g., rice, etc.), a sequence designed to improve scores for Z₃(S) may exhibit similar motifs over and over in close succession, resulting in unnaturally low complexity.

To alter the complexity of the sequence, the local sequence entropy at positions along the designed sequence (e.g., each position along the sequence, etc.) can be restrained. Local sequence entropy may be determined, by the computing device 102, using single nucleotides, dinucleotides, trinucleotides, and so forth. In one specific embodiment, the computing device 102 may be configured to calculate entropy, using dinucleotide composition, in an oligomer window of 2ω bases (2ω=128 bp) as follows in example Equation (10).

$$( 10 )$$

$${H}_{S , i} = {\sum }_{π ∈ \{ AA , AC , AG , … , TG , TT \}} \frac{‖ {S}_{n} ( i - ω , i + ω ) ‖}{2 ω} {log}_{2} ( \frac{‖ {S}_{n} ( i - ω , i + ω ) ‖}{2 ω} )$$

In Equation (10), ∥Sₙ(i−ω, i+ω)∥ is the number of occurrences of dinucleotide n in sequence S between positions i−ω and i+ω. For comparison, mean local entropy H₀ and its variance σ²_{H0} can be calculated over all sequences and all positions in A (H₀≅3.7 and σ²_{H0}≅0.03).

In some embodiments, promoters are designed based on viral promoters in the same family as 35S (Caulimoviridae). In this case, there is no obvious outgroup (B) against which to contrast the sequences. In such cases, a “simulated” background can be calculated, contrasting the frequency of a motif at a particular position in A against its average frequency across all positions in A and is defined as follows in example Equation (11).

$${Z}_{3}^{′} ( S ) = {\sum }_{i ∈ S} log ( \frac{{\sum }_{j ∈ S} ‖ {A}_{j} ‖}{‖ {A}_{i} ‖} \cdot \frac{+ \frac{‖ {A}_{i} ‖}{{\sum }_{j ∈ S} ‖ {A}_{j} ‖} ρ}{{\sum }_{j ∈ S} ‖ {A}_{j , k} ‖ + ρ} ) \qquad ( 11 )$$

The above Equation (11) may be used instead of Z₃(S) to calculate Z(S), as described above. In certain embodiments of the present disclosure, the “simulated background” method is applied even when there is an obvious outgroup B.

A score Z₄(S) that imposes a penalty on S for excessively high or low local entropy can then defined in the example Equation (12).

$${Z}_{4} ( S ) = \frac{1}{2 {σ}_{H0}^{2}} {\sum }_{i ∈ S} {( {H}_{S , i} - {H}_{0} )}^{2} \qquad ( 12 )$$

Furthermore, one skilled in the art will recognize that other measures of sequence complexity could be substituted for entropy, with similar results.

As indicated above, there are certain embodiments where it is may be desired to include certain motifs that are common in A, rather than particularly enriched relative to G. Empirically, this may provide the ability to avoid unnaturally low complexity, particularly in the case of introns, where a few motifs are strongly enriched in a relatively position-independent manner. The motif frequency score may then be defined by example Equation (13).

$${Z}_{4} ( S ) = {\sum }_{i ∈ S} log ( {4}^{k} \cdot \frac{+ {4}^{- k} ρ}{‖ {A}_{i} ‖ + ρ} ) \qquad ( 13 )$$

In Equation (13), p=1 for all work to date. This score assumes all 4^{k} possible k-mers are equally likely a priori, for example, the expected frequency of any given motif at any given position is 4^{-k}; thus, Z₅(S) is expected to be zero for a random sequence. In some cases, this can skew in the designed sequences any imbalance of A/T vs. G/C content present in the naturally occurring sequences. In such a case, the expected frequency can instead be determined separately for each k-mer based on the fraction of A, C, G, and T bases in the naturally occurring sequences.

Moreover, in addition to the above, the similarity may be further employed to modify the scoring function. For example, Z₆ is provided below, which includes a local similarity as a convolution of S with S₀ and S_{P} and a global similarity as a hamming distance similarity with the original sequence, S₀ and an optional group of sequences S_{P} which is a sequence set referenced against to avoid similarities between the sequences and the original sequence S₀. A convolution g between w and f is defined in example Equation (14).

g(x,y)=w(x,y)·f(x,y)=Σ_{s=-K/2}^{K/2}Σ_{t=-K/2}^{K/2}w(s,t)f(x+s,y+t) (14)

With additional reference to example Equation (15) below, the local similarity begins with the convolution g, and minimizing the oligomer window size K that equals the number of base pairs to avoid plus one. For example, to avoid 22 bp, then K=23.

Z₆(S,Sₒ,S_{P})=Σg(S,y)+max(H(S,y)) (15)

Finally, in various embodiments, the position-dependent k-mer enrichment score may be combined with the entropy restraint and the frequency score to obtain a final, position-dependent scoring function Z(S), as generally indicted in example Equation (16). The components are weighted by empirically determined coefficients that balance k-mer composition with sequence complexity (<φ_{z}=0.5 and ϵ_{z}=0.07 in most embodiments disclosed herein, although <φ_{z}=5 and ϵ_{z}=150 may be preferred for certain embodiments where the genetic regulatory element is an intron).

Z(S,Sₒ,S_{P})=Z₃(S)+ε_{Z}Z₄(S)+φ_{z}Z₅(S)+Z₆(S,Sₒ,S_{P}) (16)

It is expected that a promoter sequence S with a high value of Z(S) will confer a desired expression pattern on any gene of interest coupled to it.

One skilled in the art will recognize that many different techniques may be used to generate a sequence S with high values of Z(S). These techniques may include, without limitation, function optimization methods, such as simulated annealing, genetic algorithms, tabu search, simplex algorithm, steepest descent, conjugate gradients, and dynamic programming. Such methods may or may not incorporate an element of probability, randomness, or stochasticity; and may or may not involve an iterative process.

In certain embodiments of the present disclosure, the computing device 102 is configured to employ simulated annealing iteratively alter the sequence, which may be improved as defined by a score for the sequence based on the scoring function.

In particular, in the embodiment, the system 100 includes the database 104 which may include, without limitation, a motif data structure and a gene distribution data structure. The motif data structure may include a listing of motifs, which should be included in a regulatory element, or not included in the regulatory element. The motifs, in this manner, may be considered to include motifs and exclude motifs. The gene distribution data structure, similarly, may include a general list of regulatory elements, which are positive examples and negative examples. As such, the regulatory elements are intended to be more similar to the positive examples, and dissimilar to the negative examples.

The computing device 102, then, may be configured to access the database 104, including the motif data structure and the gene distribution data structure, and to generate a configuration file for generating a synthetic regulatory element. The configuration file includes, without limitation, details associated with the scoring of a synthetic regulatory element (e.g., weights, etc.) and a number of iterations, parameter schedules (e.g., temperature schedules, etc.), etc., associated with the generating of the synthetic regulatory element, etc.

In this example embodiment, the computing device 102 is configured to then initialize the scoring function, as described above (or below), consistent with the configuration file, and then to enter a first iteration of the simulated annealing operations with an initial or start sequence. Any sequence can be used as a start sequence. For example, one could use a member of set A of known expression, where there is inclusion and/or exclusion of motifs as defined in the data structure 102. It should be understood that, in this embodiment, the iterative process is based on a variance in a desired parameter (e.g., temperature in the example herein, etc.), which is a proxy for stability in this context. In general, an annealing schedule (also known as a cooling schedule, and also potentially referred to as a temperature schedule) is provided, which progresses from a high temperature to a lower temperature, thereby imposing low stability to high stability (e.g., a lower temperature may limit the number of equivalent codons, etc.) on the iterative process. The temperature schedule in this example embodiment may include, for example, {2.0, 1.0, 0.5, 0.1, 0.01}.

In particular, the computing device 102 is configured to iteratively modify the original sequence (or a start sequence for the iteration) according to a temperature of the annealing schedule. As such, the computing device 102 is configured to modify the sequence, in multiple iterations, where the temperature employed in a probability whereby the sequence is advanced to a next iteration. For example, for each iteration of a temperature, the computing device 102 is configured to score the sequence based on the scoring function Z(S, Sₒ, S_{P}) as described above, and to compare the score to the score from a prior iteration (or a score of an initial sequence for the first iteration). When the score is improved for the modified sequence, the computing device 102 is configured to pass the modified sequence into the next iteration, as the start sequence for the next iteration.

In this example embodiment, when the score of the modified sequence is not improved (based on the score), the computing device 102 is configured to use example Equation (17) below, to compare the scores of the iteration(s) (e.g., E′) to the score of the original coding sequence or prior new coding sequence (e.g., E), at the given parameter (T) herein. The computing device 102 is configured to then generate a random threshold (e.g., between 0 and 1, etc.) and to compare the probability (p) of the new coding sequence to the random threshold. When the probability satisfies the random threshold (e.g., is above the random threshold, etc.), the computing device 102 is configured to feed the new coding sequence into the next iteration as the start coding sequence. When the probability does not satisfy the random threshold, the computing device 102 is configured to discard the new coding sequence and feed the prior coding sequence into the next iteration as the start coding sequence.

$$p = exp ( - \frac{{E}^{′} - E}{T} ) \qquad ( 17 )$$

Notwithstanding the direct comparison of the resulting scores from the above equations and the specific probability function of probability equation above (for use in determining which coding sequence to implement in the next iteration), it should be appreciated that the scores may be compared otherwise in other example embodiments to make the same determination. In addition, in some example embodiments, the threshold may be a static threshold.

After each iteration at the specific temperature is completed (or stops), the computing device 102 is configured to advance to a next temperature in the annealing schedule and to repeat the iterative modification and scoring of the sequence. It should be appreciated that, in doing so in some embodiments, the computing device 102 may be configured to employ parallel processing to modify and score the coding sequence as part of the multiple different iterations at one time, whereby the iterations vary, for example, as defined by the annealing schedule. In this manner, the computing device 102 may provide a parallel tempering algorithm (e.g., in connection with replica exchange Markov chain Monte Carlo (MCMC) sampling, etc.), whereby multi-objective enhancements of the gene(s) of interest may be achieved (as generally described in connection with method 400).

Next in the system 100, after the iterations are complete for each temperature in the annealing schedule, the computing device 102 is configured to identify one or more sequences, based on the scores, as described above, as the sequence of one or more synthetic regulatory elements. The computing device 102 is configured to perform one or more checks for the output sequence(s) 108, prior to advancing to validation. For example, computing device 102 is configured to confirm the output sequence 108 includes a threshold similarity to the input sequence 106. In connection therewith, the computing device 102 may confirm that the output sequence has at most about a 75% similarity to the input sequence 106, which may be enough to avoid silencing based inclusion of the input sequence 106 and the output sequence 108 in the same stack. Apart from the overall similarity, the computing device 102 is further configured to confirm a local similarity threshold. For example, the computing device 102 may be configured to perform a stepwise comparison of the input sequence 106 and the output sequence 108 to ensure, confirm, etc. no common stretch of X base pairs exists, where X is an integer greater than 5 (e.g., X is 22 bps, etc.), etc. It should be appreciated that other checks may be perform after the iterations, whether associated with local or global similarity, or otherwise, etc.

Thereafter, the synthetic regulatory element, as defined by the sequence, is then provided to the validation phase 110, which is configured to realize and/or test the sequence. The sequence is then converted into RNA, by a person or automated process, as generally known to those skilled in the art.

And, in some embodiments, in the validation phase 110, the DNA encoding the RNA is transformed, by person or process, into a plant or plant cell by conventional means known in the art. For example, a transgene (i.e., a gene of interest) encoding the RNA of interest can be inserted into Agrobacterium, which then introduces the genetic material to plant tissue. The transformed plant tissue is then cultured in appropriate medium to promote root formation. Upon shoot formation, the transgenic plant is transferred to the appropriate soil for validation of the desired phenotype or gene of interest. In another example, a gene of interest, operably linked to the synthetic regulatory element, as defined by the sequence, can be inserted into Agrobacterium, which then introduces the genetic material to plant tissue. The transformed plant tissue is then cultured in appropriate medium to promote root formation. Upon shoot formation, the transgenic plant is transferred to the appropriate soil for validation of the ability of the generated synthetic regulatory element(s) to regulate or drive expression of the gene of interest. Alternatively, the genetic material can be introduced into a plant cell via a gene gun, electroporation, zinc finger nucleases (ZFNs), transcription activator-like effector nucleases (TALENs), the CRISPR/Cas9 system, or the CRISPR/Cpf1 system, etc. In other embodiments, in the validation phase 110, genomic DNA is edited to encode the output coding sequence.

Notwithstanding the above, it should be appreciated that the computing device 102 may be configured otherwise to employ a different manner of modifying and scoring the sequence, as indicated below.

In one example embodiment, in connection with parallel tempering, the computing device 102 is configured to initialize the input coding sequence 110 into a number of different chains, where each chain is associated with a different temperature in a given annealing schedule (or as selected randomly). The computing device 102 is configured to then initialize each chain, in parallel, for a desired number of iterations.

Specifically, for each chain, and iteration thereof, the computing device 102 is configured to alter the sequence, as limited or indicated by the given temperature selected for the chain. The computing device 102 is configured to then score the sequence, as described above. For this example embodiment, the computing device 102 is configured to then compare a score for the original coding sequence (or the prior evaluated coding sequence) and the score of the modified coding sequence, and to advance the higher scoring coding sequence (or a randomly selected lesser scoring coding sequence, as described above, based on the scoring function Z(S, Sₒ, S_{P}), above, etc.) into a next iteration. When a number of iterations is completed, the coding sequence from the last iteration is identified and advanced. Next, the computing device 102 is configured to swap the sequences for the chain with sequences from other ones of the chains, and then to continue with further iterations. The swap exposes certain sequences to alteration under a different temperature constraint, which is potentially randomly selected (or alternatively associated with the given annealing schedule).

After a defined number of iterations and swaps between chains, the computing device 102 is configured to identify one or more sequences, based on the scores, as described above, as the output sequence. The output sequence(s) 108 is then provided to the validation phase 110, wherein the output sequence(s) 108 is synthetized and tested, as described above.

Notwithstanding the above, due to the form of the scoring function, it may be suitable to employ weighted combinations (min, max, sum, etc.) of such scoring functions. The component functions may be trained on different k-mer lengths or gap structures or might be trained on different data sets. For example, a scoring function derived from genes that are relatively highly expressed in roots may be combined with a function derived from genes that are relatively highly expressed in shoots, leading to designs that should be relatively highly expressed in both roots and shoots.

In certain embodiments, multiple scoring functions are combined to retain informative parts of each function. For each k-mer and position, either the value of the most significant scoring function is used, or if no scoring function is significant, all are averaged. In certain embodiments, a position-independent approach may be used to design synthetic genetic regulatory elements or portions thereof. In other embodiments, a hybrid approach can be used where the position-dependent approach described above is employed to design a first part of the sequence of a synthetic regulatory element and a position-independent approach is employed to design a second part of the synthetic regulatory element.

The position-independent approach is based on observations concerning promoters. However, the description herein is not limited to promoters but can be used with any genetic regulatory element. For promoters, it was observed that the most significant position-specific enrichments of k-mers in promoters can occur in the approximately 200 bases prior to the TSS. Further upstream of the TSS, enrichment signals were generally weak and can be unreliable. This is consistent with the understanding in the field that there are highly position-sensitive “core promoter” elements near the TSS, and less position-specific enhancing or regulatory elements further from the TSS. Therefore, hybrid synthetic promoters were designed which optimize Z(S) in the core promoter region (about −200 to +50) and an alternative score in the upstream regulatory region (about −500 to −200). A 300 bp regulatory region was selected for experimental testing based on the sizes of naturally occurring Arabidopsis promoters, but longer or shorter regions are likely to function similarly.

In certain embodiments, including those involving genetic regulatory elements that are viral promoters, the TSS may be unknown. In such embodiments where the TSS is unknown or even in embodiments where the TSS is known, the promoters can be aligned on their TATA boxes instead. For viral promoters, for example, some signals (e.g., the TATA boxes, etc.) are so much stronger than others that it becomes difficult to choose a suitable bandwidth w for the kernel density estimation step: too little smoothing makes it difficult to detect more dispersed signals, but too much smoothing leads to tandem repeats of strong motifs like the TATA box. Thus, standard kernel density estimation can be replaced with an adaptive variant, if desired. The bandwidth is varied per motif and per position, based on the local density: weak signals are smoothed more, strong signals are smoothed less. This is expensive to compute for a large background set, and so fits particularly well with the “simulated background” approach, where only a small group of sequences needs to be processed. Alternately, adaptive KDE can be used for the in-group and fixed-bandwidth KDE can be used for the out-group, because the outgroup is highly heterogeneous, and so no sharp peaks are expected (with the possible exception of the TATA box).

FIG. 3 illustrates an example computing device 300 that may be used in the system 100. The computing device 300 may include, for example, one or more servers, workstations, personal computers, laptops, tablets, distributed computing system, embedded system, stand-alone electronic device, cloud-based platforms, mobile device, network devices, etc. In addition, the computing device 300 may include a single computing device, or it may include multiple computing devices located in close proximity or distributed over a geographic region, so long as the computing devices are specifically configured to function as described herein. In the system 100 of FIG. 1, the computing device 102 and the database 104 may include, or may be implemented in, a computing device consistent with computing device 300. That said, the system 100, or parts thereof, should not be understood to be limited to the computing device 300, as other computing device may be employed in other system embodiments. In addition, different components and/or arrangements of components may be used in other computing devices.

Further, while the computing device 102 is illustrated as a physical machine, the computing device 102 may be implemented in one or more virtual machines, or via a cloud service, whereby the operations and/or methods are implemented therein.

Referring to FIG. 3, the example computing device 300 includes a processor 302 and a memory 304 coupled to (and in communication with) the processor 302. The processor 302 may include one or more processing units (e.g., in a multi-core configuration, etc.). For example, the processor 302 may include, without limitation, a central processing unit (CPU), a microcontroller, a reduced instruction set computer (RISC) processor, an application specific integrated circuit (ASIC), a programmable logic device (PLD), a gate array, and/or any other circuit or processor capable of the functions described herein.

The memory 304, as described herein, is one or more devices that permit data, instructions, etc. to be stored therein and retrieved therefrom. The memory 304 may include one or more computer-readable storage media, such as, without limitation, dynamic random-access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable storage media. The memory 304 may be configured to store, without limitation, temperature schedules, deleterious motifs, miRNA target sites, initialization parameters, scoring functions, sequences, and/or other types of data (and/or data structures) as needed and/or suitable for use as described herein. Furthermore, in various embodiments, computer-executable instructions may be stored in the memory 304 for execution by the processor 302 to cause the processor 302 to perform one or more of the functions described herein, such that the memory 304 is a physical, tangible, and non-transitory computer readable storage media. Such instructions often improve the efficiencies and/or performance of the processor 302 that is performing one or more of the various operations herein (e.g., one or more of the operations of method 400, etc.), whereby the computing device 300 may be transformed into a special-purpose computing device. It should be appreciated that the memory 304 may include a variety of different memories, each implemented in one or more of the operations or processes described herein.

In the example embodiment, the computing device 300 includes an output device 306 (or presentation unit) that is coupled to (and that is in communication with) the processor 302 (however, it should be appreciated that the computing device 300 could include output devices other than the output device 306, etc.). The output device 306 outputs information (e.g., output coding sequences, scores, etc.), either visually or audibly, to a user of the computing device 300, for example, a user associated with the conversion engine 130, a user associated with the validation phase 110, etc. Various interfaces (e.g., as defined by network-based applications, etc.) may be displayed at computing device 300, and in particular at output device 306, to display such information. The output device 306 may include, without limitation, a liquid crystal display (LCD), a light-emitting diode (LED) display, an organic LED (OLED) display, an “electronic ink” display, speakers, etc. In some embodiments, output device 306 includes multiple devices.

The computing device 300 also includes an input device 308 that receives inputs from the user (e.g., user inputs, etc.) such as, for example, selection of a gene of interest, etc., or inputs from other computing devices. The input device 308 is coupled to (and is in communication with) the processor 302 and may include, for example, a keyboard, a pointing device, a touch sensitive panel (e.g., a touch pad or a touch screen, etc.), another computing device, and/or an audio input device. Further, in various example embodiments, a touch screen, such as that included in a tablet, a smartphone, or similar device, behaves as both output device 306 and input device 308.

In addition, the illustrated computing device 300 also includes a network interface 310 coupled to (and in communication with) the processor 302 and the memory 304. The network interface 310 may include, without limitation, a wired network adapter, a wireless network adapter, a mobile network adapter, or other device capable of communicating to/with one or more different networks. Further, in some example embodiments, the computing device 300 includes the processor 302 and one or more network interfaces incorporated into or with the processor 302.

Consistent with the above, it should be understood that the disclosure may be associated with a computer system or computer-implemented method. In general, in such embodiments, the system 100, for example, may include a source of data (e.g., one or more databases or data structures generated or made, or link to an external database, etc.), in database 104, for example, such as nucleotide sequence and/or gene expression data. The computing device 102, for example, may then include computer-executable programs or routines to process the data (e.g., software, firmware, hardware, or any combination thereof, etc.), and which configure the computing device 102 to perform the operations describe herein. The programs provide an output for either memory storage or to an output device (e.g., in the form of stored data, etc.), or to the validation phase 110, whereby the regulatory elements are realized and tested.

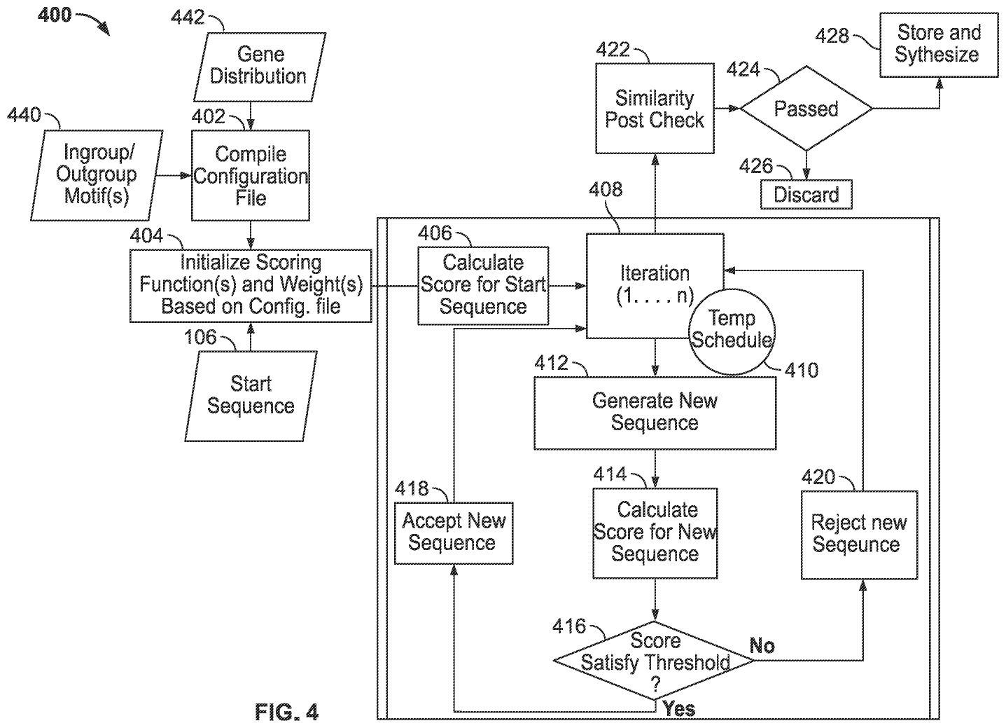

FIG. 4 illustrates an example method 400 for use in generating or identifying a synthetic regulatory element based on gene expression probability. The method 400 is described with reference to the computing device 102, and the system 100, and also with reference to the computing device 300. That said, it should be appreciated that the methods herein are not limited to the system 100, or the computing device 300, as other architectures, devices, etc., may be employed. Likewise, it should also be appreciated that the systems and/or devices herein should not be understood to be limited to the method 400, as other suitable methods may be employed in the systems and/or devices herein.

At the outset in the method 400, in this example, it should be appreciated that one or more feature extraction techniques may be employed (as generally described above in the system 100), whereby certain features of sequences associated with selected or desired gene expression properties may be identified (e.g., a list of motifs and sequence features to template in (or out) of target elements, sequence features that correlate with a target of interest (or target element if interest), etc.).

It should also be appreciated that the method 400 is provided as a simulated annealing process. In general, simulated annealing is a stochastic optimization technique that includes a stage of parameter exploration in which a large diversity of parameter configurations can be evaluated, followed by a stage of parameter exploitation in which a small set of the parameter configurations are identified that performed well (in order to further improve on the identified configurations). Through use of the exploration and exploitation stages, simulated annealing, as implemented in method 400, may enhance an algorithm's ability to efficiently converge on a desired solution.

Nonetheless, as indicated above, parallel tempering may provide an alternative approach of utilizing such exploration and exploitation stages, by simultaneously executing the stages in parallel threads and allowing the stages to exchange information about the parameter landscape they have discovered, for example, at set intervals. For instance, parallel tempering may provide N copies (or chains) of the sequence, randomly initialized at different temperatures. Each one of these copies of the sequence is referred to as a chain or replica. And, parallel tempering may make sequences as modified at higher temperatures available to lower temperature modifications, and vice versa, whereby parallel tempering may simultaneously explore low performing regions using the high temperature chains and exploits high performing regions using the low temperature chains (e.g., as a manner of refining the regions, etc.). Consequently, parallel tempering may improve the performance of the algorithm by increasing the quantity and quality of information each chain sees and/or has available thereto.

With reference specifically to FIG. 4, the method 400 is illustrated as an experiment, in which a start sequence for a regulatory element is selected, and then the method 400 is performed to get to one or more output sequences for regulatory elements. The experiment may then be repeated with the same or different start sequences, parameters, etc., as desired. A validation phase, such as illustrated in connection with FIG. 1 (e.g., validation phase 110, etc.), may be employed to determine success, prior to initiating a further experiment, etc.

Specifically, in connection with FIG. 4, at 402, the computing device 102 compiles a configuration file for the regulatory element, and the parameters associated with the regulatory element to be defined.

In particular, as shown, the computing device 102 accesses data from a motifs data structure 440, that includes both ingroup motifs and outgroups motifs, from the database 104. As explained above, the ingroup of motifs includes motifs that should be included in the sequence for the regulatory element. Likewise, the outgroup of motifs includes motifs that should not be included in the sequence for the regulatory element. The groups may include a few motifs, dozens of motifs, or, potentially, hundreds or more or less motifs, etc. In addition, the computing device 102 accesses a distribution data structure 442, which includes one or more gene distributions for the sequence, from the database 104. In particular in this example, the database 104 includes the distribution data structure 442, which generally, is specific to the type of regulatory element. As such, for example, where the regulatory element to be generated is a promoter, the gene distribution data structure 442 includes previously tested or known promotor sequences, which are desirable or undesirable. In this manner, the gene distribution data structure 442 includes a further set of ingroup regulatory elements and a set of outgroup regulatory elements, whereby the method 400 evaluates potential sequences for similarities to the sequences in the ingroup set and the outgroup set, and promotes similarities to the ingroup set and reduces (or penalizes) similarities to the outgroup set, etc.

Consistent with the above, the ingroup/outgroup data structure 440 of motifs provides a specific include/exclude of a sequence subset, while the gene distribution data structure 442 provides a more general guidelines as to the content of the sequence.

The configuration file, based on the above, may include the specific motifs, a gene distribution, and also different parameters for the simulated annealing process, such as, for example, a number of iterations, a temperature schedule, a manner of modifying the sequences (e.g., random, etc.), etc. It should be appreciated that certain parameters may be set by a user associated with the computing device 102 (e.g., technician, etc.), and certain parameters may be selected automatically (e.g., defaults, etc.), which are changeable, or not, by the user. Other parameters, such as, for example, weight values for the different terms of a scoring function, etc., may be determined through analysis determined based on user preference, mining through historical successes, machine learning methodologies, random chance, and/or any other appropriate method.

Thereafter, at 404, the computing device 102 initializes the scoring function for the experiment and applies the configuration file parameters, and a start sequence. At the outset of the experiment, the computing device 102 calculates a score for the start sequence, at 406. The scoring is based on one or more of the equations described above. In this example embodiment, the computing device 102 employs Equation (16), and therefore generates a score indicative of predicted performance of one sequence relative to one or more other sequences (during the simulated annealing process, etc.).

The computing device 102 then initiates, at 408, an iterative process, which includes n iterations, where n may be any integer greater than 1. In this example, n is 50, 100, 1,000, 10,000, 100,000, or more or less, etc.

In addition, the iterative process is set to a specific temperature (T), according to the temperature schedule 410 in a data structure (e.g., in memory 204, etc.), where the temperatures may include 10, 5, 3, 1, 0.1, 0.01 (as dimensionless units), or otherwise. With a temperature value of 10, for example, which impacts the degree of alterations in a sequence, the computing device 102 initiates, at 408, multiple iterations (e.g., iterations (i)=1 to 100, etc.) of modifying the sequence for the given temperature selection. Specifically, to start, the computing device 102 initially generates a new sequence, at 412, which is based on the start sequence and the selected N-th temperature.

In particular, in this example embodiment, the start sequence is altered by random changes in the sequence, for example, one nucleotide per iteration. Random changes may include, for example, point random position alteration, where one of the nucleotides is swapped with another at random (e.g., a point mutation in a random position, etc.). In general, the changes will involve one bp per iteration cycle, with a random position as to which bp is modified. The temperature, then, generally indicates the probability of accepting a new sequence that scores worse than the old sequence (e.g., high temperatures generally indicate an algorithm that explores more, whereby as the system cools down, it becomes less risk averse and instead prefers more and more to just improve on the current best sequence; etc.).

With continued reference to FIG. 4, after altering the sequence, the computing device 102 calculates, at 414, a sequence score for the altered sequence. To do so, the computing device 102 employs the scoring function described above to calculate the score. Thereafter, the computing device 102 determines, at 416, based on the score for the altered sequence for the iteration and the score for the input sequence (from step 406), whether the altered sequence is enhanced over the input sequence. When the score of the altered sequence is higher than the score of the input sequence, for example, the altered sequence is accepted, at 418, saved in memory (e.g., the memory 204, etc.) and returned to step 408 as the input sequence for the next iteration (e.g., i=2, etc.).

Conversely, when the score for the altered sequence is less than the score of the input sequence, the computing device 102 may reject the altered sequence, at 420, and then return to step 408 where the prior input sequence is again relied on as the input sequence in the next iteration (e.g., i=2, etc.).

Additionally, or alternatively, when the score for the altered sequence is less than the score of the input sequence, the computing device 102 may apply Equation (17) above to determine a probability between the scores, based on the temperature, and then compare the probability to a randomly generated threshold (e.g., a threshold between 0 and 1, etc.). It should be appreciated that one or more other equations (other than Equation (17)) may be employed in other embodiments to perform such an analysis. When the probability does satisfy the randomly generated threshold, it is accepted and returned to step 408 as the input sequence for the next iteration (e.g., i=2, etc.). Conversely, when the probability does not satisfy the random threshold, the new coding sequence is rejected, at 420, and the prior input coding sequence is reused as the input coding sequence for the next iteration (e.g., i=2, etc.). In this manner, an altered sequence having a lower score has the opportunity to advance as a start sequence for the next iteration, despite the lower score, thereby potentially imposing flexibility in the method 400.

When each of the iterations is complete (e.g., when i=100 in the illustrated embodiment, etc.), the computing device 102 identifies and stores an output sequence in memory (e.g., the memory 204, etc.). The computing device 102 then returns, as indicated in FIG. 4, to the iterations in step 408 and proceeds with the next temperature (e.g., the next N-th temperature, etc.) in the temperature data structure (e.g., 5, 3, 1, 0.1, 0.01, etc.), where the output sequence from the prior temperature is the start sequence for the new temperature, or alternatively, a different start sequence may be used, etc. The computing device then proceeds consistent with the above description of steps 412-420.

The steps 408-420 are repeated and completed for each of the temperatures in the temperature schedule, the computing device 102 then identifies and stores an output sequence in memory (e.g., the memory 204, etc.). The output sequence(s) are then exposed to one or more post checks. In particular, in this example embodiment, the computing device determines, at 422, whether a similarity post check is satisfied. In this example, the post check is a global similarity (e.g., is the output sequences sufficiently different from the start sequence(s) (e.g., less than 75% similarity, etc.), etc.) and a local similarity as to specific segments/chunks of the sequences being different (e.g., no stretches of 22 nucleotides in common, etc.), etc. For instance, as described, the scoring function herein penalizes sequences that have redundancies, but does not bar them outright from consideration (e.g., to avoid not considering a temporary transition between two valid sequences, etc.). The post check may be used to evaluate such reserved sequences to ensure that they in fact should be preserved. When the computing device 102 determines, at 424, that the sequence does not satisfy the post check, the new coding sequence is discarded, at 426.

Conversely, when the computing device 102 determines, at 424, that the sequence satisfy the post check, it is accepted, at 428, whereby the sequence is store in memory (e.g., the memory 304, etc.) and then synthesized consistent with the description herein.

The synthetic regulatory elements are not restricted to any particular size, but in some embodiments the sequences generated or operatively connected to genes of interest are at least 25 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 550 nucleotides, at least about 600 nucleotides, or at least about 1 kb in length.

In certain embodiments, the methods further include synthesizing a nucleic acid molecule comprising the synthetic nucleotide sequence and/or testing the nucleic acid molecule as a synthetic regulatory element to determine if the synthetic genetic regulatory element is capable of regulating gene expression of an operably linked gene of interest in the desired manner and/or in the desired cell or organism As used herein, the term “operably linked” refers to the association of nucleic acid sequences so that the function of one is regulated by the other. For example, a promoter is operably linked with a coding sequence when it is capable of regulating the expression of that coding sequence (e.g., that the coding sequence is under the transcriptional control of the promoter, etc.). Coding sequences can be operably linked to regulatory sequences in a sense or antisense orientation. In another example, the complementary RNA regions may be operably linked, either directly or indirectly, 5′ to the target mRNA, or 3′ to the target mRNA, or within the target mRNA, or a first complementary region is 5′ and its complement is 3′ to the target mRNA.

Typically, the function of genetic regulatory elements is determined by transforming the organism or at least one cell thereof with a polynucleotide construct comprising the genetic regulatory element operably linked to the gene of interest. The polynucleotide construct can further comprise additional naturally occurring or synthetic genetic regulatory elements, if desired or necessary for expression of the gene of interest in the organism or at least one cell thereof. Those of skill in the art will appreciate that determining whether the synthetic genetic regulatory element is capable of regulating the expression of an operably linked gene in the desired manner in the target organism or any other organism of interest can depend on any number of factors including, for example, the type of genetic regulatory element produced by the methods disclosed herein, the presence of additional genetic elements in the expression construct, the gene of interest to be expressed, the organism or part or cell thereof in which expression is assayed, the expression assay, the detection method (e.g., GFP visible fluorescent, detection of GFP RNA by qPCR), the environmental conditions during the assay, and the like.

For example, in certain embodiments in which the synthetic genetic regulatory element is a promoter and expression of the gene of interest is evaluated by expression of the encoded protein, about 5-15% of the genetic regulatory elements produced by the methods herein may display expression detectable by confocal imaging of GFP fluorescence in Arabidopsis thaliana in the T1 generation in the absence of an enhancing intron in the polynucleotide construct. However, when the polynucleotide construct further comprises an enhancing intron about 60% of the synthetic genetic regulatory elements display detectable expression by confocal imaging of GFP fluorescence in the T1 generation, when assayed in Arabidopsis thaliana by the methods disclosed herein. Similarly, when promoter activity is determined at the nucleic acid level, for example, by sensitive qPCR detection, about 60% of the genetic regulatory elements display detectable promoter activity without the addition of an enhancing intron. These results indicate that the majority of synthetic promoters produced by the methods in the present disclosure have biological promoter activity in plants.

In determining whether the synthetic genetic regulatory element is capable of regulating the expression of an operably linked gene in the desired manner, a reporter gene may be employed. As used herein a “reporter” or a “reporter gene” refers to a nucleic acid molecule encoding a detectable marker. Preferred reporter genes include, for example, luciferase (e.g., firefly luciferase or Renilla luciferase, etc.), β-galactosidase, chloramphenicol acetyl transferase (CAT), and a fluorescent protein (e.g., green fluorescent protein (GFP), red fluorescent protein (DsRed), yellow fluorescent protein, blue fluorescent protein, cyan fluorescent protein, or variants thereof, including enhanced variants such as enhanced GFP (eGFP), etc.). Reporter genes are detectable by a reporter assay. Reporter assays can measure the level of reporter gene expression or activity by any number of means, including, for example, measuring the level of reporter mRNA, the level of reporter protein, or the amount of reporter protein activity. Reporter assays are known in the art or otherwise disclosed herein.

The synthetic genetic regulatory elements that are produced as described herein are not limited to use in the target organism from which the one or more sets of genes as described herein were derived. In one example, a genetic regulatory element that is produced as described herein using a first set of nucleotide sequences of a genetic regulatory element from Arabidopsis thaliana finds use in regulating the expression of an operably linked gene of interest in an Arabidopsis thaliana plant, a soybean plant, and/or in one or more other dicotyledonous plants of interest. In another example, a synthetic genetic regulatory element that is produced as described herein using a first set of nucleotide sequences of a genetic regulatory element from rice finds use in regulating the expression of an operably linked gene of interest in a rice plant, a maize plant, and/or in one or more other monocotyledonous plants of interest. In yet another example, a synthetic genetic regulatory element that is produced as described herein using a first set of nucleotide sequences of a genetic regulatory element from Caulimoviridae viruses finds use in regulating the expression of an operably linked gene of interest in an Arabidopsis thaliana plant, a soybean plant, a rice plant, a maize plant, and/or in one or more other monocotyledonous and/or dicotyledonous plants of interest.

In some embodiments, the synthetic regulatory element is a promoter. “Promoter” refers to a nucleic acid that is capable of controlling the expression of an operably linked coding sequence or other sequence encoding an RNA that is not necessarily translated into a protein. The promoter sequence may include proximal and more distal upstream elements, the latter elements often referred to as enhancers. An “enhancer” is a DNA sequence that may stimulate promoter activity and may be an innate element of the promoter or a heterologous element inserted to enhance the level or tissue-specificity of a promoter. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, nucleic acid fragments of some variation may have identical promoter activity.

In some embodiments, the promoter is a plant promoter. A “plant promoter” is a promoter capable of initiating transcription in plant cells whether or not its origin is a plant cell. For example, it is well known that Agrobacterium promoters are functional in plant cells. Thus, plant promoters include promoter DNA obtained from plants, plant viruses and bacteria such as Agrobacterium and Bradyrhizobium bacteria, and synthetic promoters capable of initiating transcription in plant cells. A plant promoter can be a constitutive promoter, a non-constitutive promoter, an inducible promoter, a repressible promoter, a tissue specific promoter (e.g., a root specific promoter, a stem specific promoter, a leaf specific promoter, an ovary specific promoter, an anther specific promoter, a tassel specific promoter, etc.), a tissue preferred promoter (e.g., a root preferred promoter, a stem preferred promoter, a leaf preferred promoter, an ovary preferred promoter, an anther preferred promoter, a tassel preferred promoter, etc.), a cell type specific or preferred promoter (e.g., a meristem cell specific/preferred promoter, an egg cell specific/preferred promoter, a pollen specific/preferred promoter, etc.), or any other type.

A constitutive promoter is a promoter which is active under most conditions and/or during most development stages. There may be certain advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. For illustration, constitutive promoters include, CaMV 35S promoter, opine promoters, ubiquitin promoter, actin promoter, alcohol dehydrogenase promoter, etc. In some embodiments, the synthetic promoter prepared as described herein, is used to drive expression of a heterologous sequence, while CaMV 35S promoter is used to drive expression of a second sequence.

A non-constitutive promoter is a promoter which is active under certain conditions, in certain types of cells, and/or during certain development stages. For example, tissue specific or preferred, cell type specific or preferred, inducible promoters, and promoters under developmental control are non-constitutive promoters. Examples of promoters under developmental control include promoters that preferentially initiate transcription in certain tissues, such as stems, leaves, roots, embryos, or seeds.

An “inducible” or “repressible” promoter is a promoter which is under chemical or environmental factor control. Examples of environmental conditions that may affect transcription by inducible promoters include cold, heat, drought, light, or certain chemicals.

A “tissue specific” promoter is a promoter that initiates transcription only in certain tissues. Unlike constitutive expression of genes, tissue-specific expression is the result of several interacting levels of gene regulation. As such, sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature. Non-limiting tissue specific promoters include, beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression), and embryo specific promoters, e.g., a promoter associated with an ammo acid permease gene (AAP1), an oleate 12-hydroxylase:desaturase gene from Lesquerellafendleri (LFAH12), an 2S2 albumin gene (2S2), a fatty acid elongase gene (FAE1), or a leafy cotyledon gene (LEC2). For example, a “root specific” promoter is a promoter that initiates transcription only in root tissues.

A “tissue preferred” promoter is a promoter that initiates transcription mostly, but not necessarily entirely or solely in certain tissues. For example, a “root preferred” promoter is a promoter that initiates transcription mostly, but not necessarily entirely or solely in root tissues.

A “cell type specific” promoter is a promoter that primarily drives expression in certain cell types in one or more organs, for example, vascular cells in roots, leaves, stalk cells, and stem cells.

A “cell type preferred” promoter is a promoter that primarily drives expression mostly, but not necessarily entirely or solely in certain cell types in one or more organs, for example, vascular cells in roots, leaves, stalk cells, or stem cells.

In some embodiments, the synthetic regulatory element is an expression-enhancing intron. An “expression-enhancing intron” or “enhancing intron” is an intron that is capable of causing an increase in the expression of a gene to which it is operably linked.

In some embodiments, the synthetic regulatory element is a transcription termination region (or 3′UTR). As used herein, the terms “3′ transcription termination molecule,” “3′ untranslated region” or “3′UTR” refer to a DNA molecule that is used during transcription to the untranslated region of the 3′ portion of an mRNA molecule. The 3′ untranslated region of an mRNA molecule may be generated by specific cleavage and 3′ polyadenylation, also known as a polyA tail. A 3′UTR may be operably linked to and located downstream of a transcribable DNA molecule (e.g., a gene of interest etc.) and may include a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region, wheat hsp17 3′ region, pea rubisco small subunit 3′ region, cotton E6 3′ region, and the coixin 3′ UTR. 3′ UTRs typically find beneficial use for the recombinant expression of specific DNA molecules.

In other aspects, the disclosure herein provides for making expression vectors, transgenic cells, or non-human transgenic organisms, as described herein for producing synthetic regulatory elements. The disclosure involves operably linking a synthetic regulatory element produced as described herein to a gene of interest so as to produce an expression construct. Such genes of interest will depend on the desired outcome and can comprise nucleotide sequences that encode proteins and/or RNAs of interest. Nucleic acid molecules can then be synthesized or produced using a number of methods known in the art. These include chemical synthesis and recombinant techniques. The disclosure herein may further involve transforming at least one cell with the polynucleotide construct. The disclosure can additionally involve propagating the cell or regenerating a transgenic organism from the transformed cell.

As used herein, the phrases “recombinant construct”, “expression construct”, “chimeric construct”, “construct”, and “recombinant DNA construct” are used interchangeably. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source but arranged in a manner different than that found in nature. Such construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the present disclosure. Screening transformants may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. Where appropriate, the genes of interest may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, sulfonylurea, glyphosate, glufosinate, L-phosphinothricin, triazine, benzonitrile and 2,4-dichlorophenoxyacetate (2,4-D). Additional selectable markers include phenotypic markers such as galactosidase and fluorescent proteins such as green fluorescent protein (GFP), cyan florescent protein (CYP), yellow florescent protein, etc.

One of ordinary skill in the art will recognize that the systems and methods for use in generating synthetic regulatory elements are applicable to organisms other than plants. In certain aspects, the disclosure herein provides for making a transgenic cell or non-human organism, by incorporating a synthetic regulatory element in operable association with a coding sequence or other transcribed gene into one or more cells, where the synthetic regulatory element has a statistically significant score with the scoring function described herein. The cells are propagated to make the transgenic cell or non-human organism. It is recognized that the genetic regulatory elements of the present disclosure and expression cassettes comprising one or more of such genetic regulatory elements can be used for the expression in both human and non-human host cells. In certain aspects, the disclosure herein provides for making a pluripotent stem cell, by incorporating a synthetic regulatory element in operable association with a coding sequence or other transcribed gene into one or more pluripotent stem cells, where the synthetic regulatory element has a statistically significant score with the scoring function described herein. In one embodiment of the present disclosure, the host cells are human host cells or a host cell line that is incapable of differentiating into a human being.

The disclosure may further involve introducing a polynucleotide construct into a plant. The term “introducing” means presenting to the plant the polynucleotide construct in such a manner that the construct gains access to the interior of a cell of the plant. The disclosure does not depend on a particular method for introducing a polynucleotide construct to a plant, only that the polynucleotide construct gains access to the interior of at least one cell of the plant. The transformation may be stable or transient.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

Suitable methods of introducing nucleotide sequences into plant cells and subsequent insertion into the plant genome may include, for example, microinjection as electroporation, direct gene transfer, and ballistic particle acceleration, etc.

The polynucleotides herein may be introduced into plants by contacting plants with a virus or viral nucleic acids. Generally, such methods involve incorporating a polynucleotide construct within a viral DNA or RNA molecule. Further, it is recognized that promoters produced as described herein also encompass promoters utilized for transcription by viral RNA polymerases.

The cells that have been transformed may be grown into plants in accordance with conventional techniques. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruits, roots, root tips, anthers, and the like. Progeny, variants, and mutants of the regenerated plants are also included within the scope of the present disclosure, provided that these parts comprise the introduced polynucleotides (e.g., comprising the synthetic regulatory element, etc.).

With respect particularly to plants, genes of interest that are controlled by the synthetic regulatory element are reflective of the markets and/or interests of those involved in the development of the crop. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, yield, abiotic stress tolerance, and commercial products. Genes of interest include, generally, those involved in oil, starch, carbohydrate, or nutrient metabolism. In addition, genes of interest include genes encoding enzymes and other proteins from plants and other sources including prokaryotes and other eukaryotes.

In certain embodiments, the disclosure relates to transgenic plants and methods for making the same. As used herein, the term “plant” refers to any living organism belonging to the kingdom Plantae (e.g., any genus/species in the Plant Kingdom, etc.). In some embodiments, the plant is a tree, herb, bush, grass, vine, fem, moss, or green algae. The plant may be monocotyledonous (monocot) or dicotyledonous (dicot). Examples of particular plants include but are not limited to Arabidopsis, Brachypodium, switchgrass, corn, potato, rose, apple tree, sunflower, wheat, rice, banana, tomato, opo, pumpkin, squash, lettuce, cabbage, oak tree, guzmania, geranium, hibiscus, clematis, poinsettia, sugarcane, taro, duck weed, pine tree, Kentucky blue grass, zoysia, coconut tree, cauliflower, cavalo, collard, kale, kohlrabi, mustard greens, rape greens, and other brassica leafy vegetable crops, bulb vegetables (e.g., garlic, leek, onion (dry bulb, green, and Welch), shallot, etc.), citrus fruits (e.g., grapefruit, lemon, lime, orange, tangerine, citrus hybrids, pummelo, and other citrus fruit crops, etc.), cucurbit vegetables (e.g., cucumber, citron melon, edible gourds, gherkin, muskmelons (including hybrids and/or cultivars of cucumis melons), water-melon, cantaloupe, etc.), fruiting vegetables (including eggplant, ground cherry, pepino, pepper, tomato, tomatillo), grape, leafy vegetables (e.g., romaine, etc.), root/tuber and corm vegetables (e.g., potato, etc.), and tree nuts (almond, pecan, pistachio, and walnut), berries (e.g., tomatoes, barberries, currants, elderberries, gooseberries, honeysuckles, mayapples, nannyberries, Oregon-grapes, buckthorns, hackberries, bearberries, lingonberries, strawberries, sea grapes, b lackberries, cloudberries, loganberries, raspberries, salmonberries, thimbleberries, and wineberries, etc.), cereal crops (e.g., corn (maize), rice, wheat, barley, sorghum, millets, oats, ryes, triticales, buckwheats, fonio, quinoa, oil palm, etc.), Brassicaceae family plants, and Fabaceae family plants, pome fruit (e.g., apples, pears, etc.), stone fruits (e.g., coffees, jujubes, mangos, olives, coconuts, oil palms, pistachios, almonds, apricots, cherries, damsons, nectarines, peaches and plums, etc.), vine (e.g., table grapes, wine grapes, etc.), fiber crops (e.g., hemp, cotton, etc.), ornamentals, and the like.

In some embodiments, nucleic acid molecules are introduced into a plant by cloning the nucleic acid molecules into a binary vector suitable for plant-specific transformation. For example, to introduce the nucleic acid molecules in Brassica species, nucleic acid molecules are cloned into a binary vector suitable for Brassica species transformation.

In certain embodiments, the plant is a cultivar. As used herein, the term “cultivar” refers to a variety, strain or race of plant that has been produced by horticultural or agronomic techniques and is not normally found in wild populations.

The disclosure in certain aspects includes plant parts derived from the transgenic plants described herein. As used herein, the term “plant part” refers to any part of a plant including but not limited to the shoot, root, stem, stalk, trunk, tiller, seeds, endosperm, pedicel, tuber, rhizomes, stipules, stolon, nodules, leaves or leaf sheath, needle, cone, petals, flowers, ovules, fruit, berry, stigma, bracts, peduncle, branches, style, carpel, pericarp, petioles, internodes, bark, pubescence, pollen, stamen, pistil, sepal, anther, placenta, and the like. The two main parts of plants grown in some sort of media, such as soil, are often referred to as the “above-ground” part, also often referred to as the “shoots”, and the “below-ground” part, also often referred to as the “roots”.

In some embodiments, the disclosure provides for making a transgenic plant having a gene of interest under the control of a synthetic promoter that is operable, for example, in rice. The transgenic plant may or may not be a species of rice. And, the synthetic promoter may be a high constitutive promoter.

In some embodiments, the present disclosure provides a method of making a transgenic plant having a gene of interest under the control of a synthetic promoter. And, the synthetic promoter may be a constitutive promoter.

In some embodiments, the present disclosure provides for making a transgenic plant having a gene of interest under the control of a synthetic promoter. And, the synthetic promoter may be a high constitutive promoter.

In some embodiments, the present disclosure provides a method of making a transgenic plant having a gene of interest operably associated with a synthetic intron. And, the synthetic intron may be an expression enhancing intron.

In some embodiments, the transgenic plant has a gene of interest under control of a synthetic promoter and synthetic intron as described above.

The disclosure herein can be used in connection with basic plant breeding techniques. For example, the transgenic plant may be inbred or a single allele converted plant. As used herein, the term “inbred” or “inbred plant” includes any single gene conversions of that inbred. The phrase “single allele converted plant” refers to those plants which are developed by a plant breeding technique called backcrossing wherein essentially all of the desired morphological and physiological characteristics of an inbred are recovered in addition to the single allele transferred into the inbred via the backcrossing technique. In some embodiments, an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids. Developing the transgenic plants may further include crossing. As used herein, the term “cross”, “crossing”, “cross pollination” or “cross-breeding” refer to the process by which the pollen of one flower on one plant is applied (artificially or naturally) to the ovule (stigma) of a flower on another plant.

In certain embodiments, the disclosure involves transformation of cells. As used herein, the term “transformant” refers to a cell, tissue or organism that has undergone transformation. The original transformant may be designated as “T0” or “T₀”. Selfing the TO produces a first transformed generation designated as “T1” or “T₁”.

In some embodiments, the transgenic cell or organism is hemizygous for the gene of interest under control of the synthetic regulatory element. As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

In some embodiments, the cell or organism is heterozygous for the gene of interest under control of the synthetic regulatory element. As used herein, the term “heterozygote” refers to a diploid or polyploid individual cell or plant having different alleles (forms of a given gene) present at least at one locus. Similarly, the term “heterozygous” refers to the presence of different alleles (forms of a given gene) at a particular gene locus. In other embodiments, the cell or organism is a homozygote for the gene of interest under control of the synthetic element. As used herein, the term “homozygote” refers to an individual cell or plant having the same alleles at one or more loci. Thus, the term “homozygous” refers to the presence of identical alleles at one or more loci in homologous chromosomal segments.

Any transgenic plant comprising one or more synthetic promoters and/or synthetic introns may be used as a donor to produce more transgenic plants through plant breeding methods well known to those skilled in the art. The goal in general is to develop new, unique and superior varieties and hybrids. In some embodiments, selection methods, e.g., molecular marker assisted selection, can be combined with breeding methods to accelerate the process.

In some embodiments, example methods may include (i) crossing any one of the plants of the present disclosure comprising one or more synthetic promoters and/or synthetic introns as a donor to a recipient plant line to create a F1 population; (ii) evaluating the transgene expression in the offspring derived from said F1 population; and (iii) selecting offspring that have functional transgene expression under the control of the synthetic promoters and/or synthetic introns.

In some embodiments, complete chromosomes of the donor plant are transferred. For example, the transgenic plant with the synthetic promoters and/or synthetic introns can serve as a male or female parent in a cross pollination to produce offspring plants, wherein by receiving the transgene from the donor plant, the offspring plants obtained the synthetic promoters and/or synthetic introns. In some embodiments, only the genomic fragment containing the transgene (e.g., having the synthetic promoters and/or synthetic introns, etc.) is incorporated into the recipient plant.

The disclosure further provides for developing plants in a plant breeding program using plant breeding techniques including recurrent selection, backcrossing, pedigree breeding, molecular marker (Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Simple Sequence Repeats (SSRs) which are also referred to as Microsatellites, etc.) enhanced selection, genetic marker enhanced selection and transformation. Seeds, plants, and parts thereof produced by such breeding methods are also part of the present disclosure.

In connection therewith, several embodiments relate to a recombinant nucleic acid comprising one or more output coding sequences. As used herein, a “recombinant nucleic acid” refers to a nucleic acid molecule (DNA or RNA) having a coding and/or non-coding sequence distinguishable from endogenous nucleic acids found in natural systems. In some aspects, a recombinant nucleic acid provided herein is used in any composition, system or method provided herein. In some aspects, a recombinant nucleic acid provided herein is a transgene. In some aspects, a recombinant nucleic acid may encode any protein and can be used in any composition, system or method provided herein. In some embodiments, a recombinant nucleic acid comprises one or more output coding sequences operably linked to a heterologous promoter. In one aspect, a recombinant nucleic acid provided herein comprises one or more, two or more, three or more, four or more, five or more, six or more, seven or more, eight or more, nine or more, or ten or more heterologous promoters operably linked to one or more, two or more, three or more, four or more, five or more, six or more, seven or more, eight or more, nine or more, or ten or more output coding sequences.

In some aspects, a recombinant nucleic acid may be contained in a vector. As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular, etc.); nucleic acid molecules that comprise DNA, RNA, or both; and other varieties of polynucleotides known in the art. One type of vector is an Agrobacterium T-DNA. Another type of vector is a viral vector, wherein virally-derived DNA or RNA sequences are present in the vector for packaging into a virus (e.g., retroviruses, replication defective retroviruses, Tobacco mosaic virus (TMV), Potato virus X (PVX) and Cowpea mosaic virus (CPMV), tobamovirus, Gemini viruses, adenoviruses, replication defective adenoviruses, and adeno-associated viruses, etc.). Viral vectors also include polynucleotides carried by a virus for transfection into a host cell. In some embodiments, a viral vector may be delivered to a plant using Agrobacterium. Certain vectors are capable of autonomous replication in a host cell into which they are introduced. Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein. In some embodiments, an expression vector can comprise one or more output coding sequences in a form suitable for expression of the output coding sequences in a plant cell, which means that the expression vector comprises one or more regulatory elements that are operatively-linked to the output coding sequences to be expressed. Regulatory elements may include enhancers, termination sequences, introns, etc.

In an aspect, a vector provided herein comprises any recombinant nucleic acid comprising an output coding sequence as provided herein. In another aspect, a plant cell provided herein comprises a recombinant nucleic acid provided herein. In another aspect, a plant cell provided herein comprises a vector provided herein. The plant cell may be of a monocotyledonous plant or dicotyledonous plant. In some embodiments, the plant cell may be from or of a crop or grain plant such as cassava, corn, sorghum, alfalfa, cotton, soybean, canola, wheat, oat or rice. The plant cell may also be of an algae, tree or production plant, fruit or vegetable (e.g., trees such as citrus trees, (e.g., orange, grapefruit or lemon trees; peach or nectarine trees; apple or pear trees; etc.); nut trees such as almond or walnut or pistachio trees; nightshade plants; plants of the genus Brassica; plants of the genus Lactuca; plants of the genus Spinacia; plants of the genus Capsicum; cotton, tobacco, asparagus, avocado, papaya, cassava, carrot, cabbage, broccoli, cauliflower, tomato, eggplant, pepper, lettuce, spinach, strawberry, potato, squash, melon, blueberry, raspberry, blackberry, grape, coffee, cocoa, etc.).

That said, and as generally described above, numerous methods for transforming chromosomes or plastids in a plant cell with a recombinant DNA molecule are known in the art, which can be used according to the systems and methods of the present application to produce a plant cell and plant comprising one or more output coding sequences (e.g., at the validation phase 110, etc.).

In planta, particle bombardment or biolistic delivery can be used for delivering recombinant nucleic acids. Particle bombardment is suitable to transform plants with DNA, RNA, protein, or any combinations thereof. Methods of transforming plants using biolistic delivery of DNA is described in PCT/US2019/033984 and incorporated by reference herein, in its entirety.

In planta, Agrobacterium mediated transformation is a suitable method of choice for delivering recombinant nucleic acids on one or more T-DNAs. Agrobacterium mediated transformation is widely applied to monocot and dicot species. The expression cassettes comprising one or more output coding sequences may be provided, in one embodiment, as double tumor-inducing (Ti) plasmid border constructs that have the right border (RB or AGRtu.RB) and left border (LB or AGRtu.LB) regions of the Ti plasmid isolated from Agrobacterium tumefaciens comprising a T-DNA that, along with transfer molecules provided by the A. tumefaciens cells, permit the integration of the T-DNA into the genome of a plant cell (see, e.g., U.S. Pat. No. 6,603,061, which is incorporated herein by reference in its entirety). The constructs may also contain the plasmid backbone DNA segments that provide replication function and antibiotic selection in bacterial cells, e.g., an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. In some embodiments, one or more expression cassettes comprising one or more output coding sequences are provided in a T-DNA binary vector that has a low copy origin of replication, such as the OriRi vector backbone. For plant transformation, the host bacterial strain is often A. tumefaciens ABI, C58, or LBA4404, however other strains known to those skilled in the art of plant transformation can function in the present disclosure. In some embodiments, an Agrobacterium tumefaciens strain that lacks certain DNA recombination functions, such as RecA, is utilized to deliver expression vectors encoding CAST system components to plant cells.

In some embodiments, the expression cassettes comprising one or more output coding sequences as described herein are provided on a single T-DNA. In some embodiments, the expression cassettes encoding one or more output coding sequences as described herein are provided on multiple separate T-DNAs and delivered to plant cells in a single transformation process, or in separate sequential transformation processes.

Several embodiments relate to a plant comprising in its genome an output coding sequence as described herein. In certain embodiments, genome editing methods are utilized for the modification or replacement of an existing genomic coding sequence, such as a coding sequence for a protein conferring herbicide tolerance, within a plant genome with a sequence encoding an output coding sequence as provided by the methods described herein. In some embodiments, the native genomic coding sequence is modified to comprise one or more targeted nucleotide changes, additions, deletions, or other modifications to introduce an output coding sequence as provided by the methods described herein. Several embodiments relate to the use of a known genome editing methods, and a site-specific genome modification enzyme, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system) to modify or replace an existing coding sequence in the genome of a plant. Several embodiments relate to providing a site-specific genome modification enzyme capable of recognizing a specific gene of interest within a genome of a plant to allow for alteration of the native sequence by non-templated editing or by templated editing to introduce an output coding sequence as provided by the methods described herein. Several embodiments relate to providing a base modification agent (such as a deaminase) linked to a site-specific genome modification enzyme capable of recognizing a specific nucleotide sequence of interest within a genome of a plant to allow for alteration of the native sequence by the base modification agent to provide the output coding sequence.

Again, and as previously described, it should be appreciated that the functions described herein, in some embodiments, may be described in computer executable instructions stored on a computer readable media, and executable by one or more processors. The computer readable media is a non-transitory computer readable storage medium. By way of example, and not limitation, such computer-readable media can include RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the operations described herein and/or recited in the corresponding claims hereinafter. For example, the technical effect may be achieved by: (a) identifying an input sequence associated with a regulatory element as a start sequence; (b) calculating a score for the start sequence, based on a scoring function; (c) initializing N iteration(s) at at least one parameter (e.g., temperature, etc.), where N is an integer; (d) for each of the N iterations: (i) altering at least one nucleotide in an input sequence for the iteration; (ii) calculating a score for the altered sequence based on the scoring function; (iii) advancing the altered sequence to a next iteration based on at least the calculated score for the altered sequence and a threshold; and (iv) identifying the altered sequence as an output sequence for the N iterations when the calculated score indicates an enhancement over the input coding sequence and the iteration is equal to N; and (e) after the N iterations, directing the output sequence to a validation phase, whereby the sequence defining the regulatory element is synthesized.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (e.g., the disclosure of a first value and a second value for a given parameter can be interpreted as disclosing that any value between the first and second values could also be employed for the given parameter, etc.). For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and the phrase “at least one of” include any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for use in identifying regulatory elements, the computer-implemented method comprising:
identifying, by a computing device, an input sequence associated with a regulatory element as a start sequence;
calculating, by the computing device, a score for the start sequence, based on a scoring function;
initializing N iteration(s) at at least one parameter, where N is an integer;
for each of the N iterations:
altering at least one nucleotide in an input sequence for the iteration;
calculating a score for the altered sequence based on the scoring function;
advancing the altered sequence to a next iteration based on at least the calculated score for the altered sequence and a threshold; and
identifying the altered sequence as an output sequence for the N iterations when the calculated score for the altered sequence indicates an enhancement over the input sequence and the iteration is equal to N; and then
after the N iterations, directing the output sequence to a validation phase, whereby the sequence defining the regulatory element is synthesized.

2. The computer-implemented method of claim 1, wherein advancing the altered sequence to a next iteration is based on the calculated score for the altered sequence being greater than the calculated score for the start sequence.

3. The computer-implemented method of claim 2, wherein advancing the altered sequence to a next iteration is further based on the iteration being less than N.

4. The computer-implemented method of claim 1, wherein advancing the altered sequence to a next iteration is further based on a probability function.

5. The computer-implemented method of claim 1, further comprising discarding the altered sequence in response to the calculated score for the altered sequence being less than the calculated score for the start sequence.

6. The computer-implemented method of claim 1, wherein the regulatory element includes one or more of a promotor, an intron, and/or an untranslated region (UTR).

7. The computer-implemented method of claim 1, wherein calculating the score for the altered sequence includes calculating the score based on:
Z(S,Sₒ,S_{P})=Z₃(S)+ε_{Z}Z₄(S)+φ_{z}Z₅(S)+Z₆(S,Sₒ,S_{P}).

8. The computer-implemented method of claim 1, wherein advancing the altered sequence to the next iteration, based on a probability function satisfying the threshold and the iteration being less than N; and
wherein the threshold includes one of a static threshold and a randomly generated threshold per iteration.

9. The computer-implemented method of claim 8, wherein the probability function includes:
$$p = exp ( - \frac{{E}^{′} - E}{T} ) ;$$
and
wherein E is the calculated score of the start sequence, E′ is the calculated score of the altered sequence, and T is the at least one parameter.

10. The computer-implemented method of claim 1, wherein N is less than 100; and/or
wherein the at least one parameter is temperature.

11. The computer-implemented method of claim 1, further comprising synthesizing the regulatory element defined by the output sequence.

12. A non-transitory computer-readable storage medium including executable instructions for identifying regulatory elements, which when executed by at least one processor, cause the at least one processor to:
identify an input sequence associated with a regulatory element as a start sequence;
calculate a score for the start sequence, based on a scoring function;
initialize N iteration(s) at at least one parameter, where N is an integer;
for each of the N iterations:
alter at least one nucleotide in an input sequence for the iteration;
calculate a score for the altered sequence based on the scoring function;
advance the altered sequence to a next iteration based on at least the calculated score for the altered sequence and/or a threshold; and
identify the altered sequence as an output sequence for the N iterations when the calculated score for the altered sequence indicates an enhancement over the input sequence and the iteration is equal to N; and then
after the N iterations, direct the output sequence to a validation phase, whereby the output sequence defining the regulatory element is synthesized.

13. The non-transitory computer-readable storage medium of claim 12, wherein the executable instructions, when executed by the at least processor to advance the altered sequence to a next iteration, cause the at least one processor to advance the altered sequence to the next iteration based on the calculated score for the altered sequence being greater than the calculated score for the start sequence and the iteration being less than N.

14. The non-transitory computer-readable storage medium of claim 13, wherein the regulatory element includes one or more of a promotor, an intron, and/or an untranslated region (UTR).

15. The non-transitory computer-readable storage medium of claim 14, wherein the executable instructions, when executed by the at least processor to calculate the score for the altered sequence, cause the at least one processor to calculate the score based on:
Z(S,Sₒ,S_{P})=Z₃(S)+ε_{Z}Z₄(S)+φ_{z}Z₅(S)+Z₆(S,Sₒ,S_{P})

16. The non-transitory computer-readable storage medium of claim 12, wherein the executable instructions, when executed by the at least processor to advance the altered sequence to the next iteration, cause the at least one processor to advance the altered sequence to the next iteration further based on a probability function satisfying the threshold and the iteration being less than N;
wherein the threshold includes one of a static threshold and a randomly generated threshold per iteration;
wherein the probability function includes:
$$p = exp ( - \frac{{E}^{′} - E}{T} ) ;$$
and
wherein E is the calculated score of the start sequence, E′ is the calculated score of the altered sequence, and T is the at least one parameter.

17. The non-transitory computer-readable storage medium of claim 16, wherein the executable instructions, when executed by the at least processor to calculate the score for the altered sequence, cause the at least one processor to calculate the score based on:
Z(S,Sₒ,S_{P})=Z₃(S)+ε_{Z}Z₄(S)+φ_{z}Z₅(S)+Z₆(S,Sₒ,S_{P})

18. A system for use in identifying regulatory elements, the computer-implemented system comprising:
at least one computing device configured, by executable instructions, to:
identify an input sequence associated with a regulatory element as a start sequence;
calculate a score for the start sequence, based on a scoring function;
initialize N iteration(s) at at least one parameter, where N is an integer;
for each of the N iterations:
alter at least one nucleotide in an input sequence for the iteration;
calculate a score for the altered sequence based on the scoring function;
advance the altered sequence to a next iteration based on at least the calculated score for the altered sequence and a threshold; and
identify the altered sequence as an output sequence for the N iterations when the calculated score for the altered sequence indicates an enhancement over the input sequence and the iteration is equal to N; and
a validation phase configured to synthesize the regulatory element defined by the output sequence, after the N iterations.

19. The system of claim 18, wherein the computing device is configured, in order to advance the altered sequence to a next iteration, to advance the altered sequence to the next iteration based on the calculated score for the altered sequence being greater than the calculated score for the start sequence and the iteration being less than N.

20. The system of claim 19, wherein the computing device is configured, in order to calculate the score for the altered sequence, to calculate the score based on:
Z(S,Sₒ,S_{P})=Z₃(S)+ε_{Z}Z₄(S)+φ_{z}Z₅(S)+Z₆(S,Sₒ,S_{P})

21. The system of claim 20, wherein the computing device is configured, in order to advance the altered sequence to the next iteration, to advance the altered sequence to the next iteration further based on a probability function satisfying the threshold;
wherein the threshold includes one of a static threshold and a randomly generated threshold per iteration;
wherein the probability function includes:
$$p = exp ( - \frac{{E}^{′} - E}{T} ) ;$$
and
wherein E is the calculated score of the start sequence, E′ is the calculated score of the altered sequence, and T is the at least one parameter.

22. The system of claim 21, wherein the regulatory element includes one or more of a promotor, an intron, and/or an untranslated region (UTR);
wherein N is less than 100; and/or
wherein the at least one parameter is temperature.
